(19) United States
(12) Patent Application Publication
(10) Pub. No.: US 2012/0134986 A1
(43) Pub. Date: May 31, 2012

(54) Methods of Prognosis for Non-Hodgkin Lymphoma

(76) Inventors: Arash Ash Alizadeh, San Mateo, CA (US); Ronald Levy, Stanford, CA (US); Andrew J. Gentles, Menlo Park, CA (US); Izidore S. Lossos, Miami, FL (US)

(21) Appl. No.: 13/253,491

(22) Filed: Oct. 5, 2011

Related U.S. Application Data
(60) Provisional application No. 61/404,616, filed on Oct. 5, 2010.

(51) Int. Cl. A61K 39/395; A61P 35/00; A61K 31/4375; A61K 31/56; A61K 31/66; A61K 31/704

(52) U.S. Cl. 424/133.1; 514/110; 514/34; 514/283; 514/179

(57) ABSTRACT

Measurement of a single gene expressed by tumor cells (LMO2) and a single gene expressed by the immune microenvironment (TNFRSF9), which determination may be referred to herein as a two gene score (TGS), powerfully predicts overall survival in patients with NHL, particularly overall survival in the context of anthracycline-based chemotherapy or co-treatment with anthracycline-based chemotherapy and anti-CD20 immunotherapy. It is shown herein that increased levels of LMO2 and TNFRSF9 correlate with a positive patient response and improved prognosis.

### FEDERALLY SPONSORED RESEARCH AND DEVELOPMENT

This invention was made with Government support under contract CA034233 awarded by the National Institutes of Health. The Government has certain rights in this invention.

### BACKGROUND

Non-Hodgkin lymphomas are a heterogeneous group of disorders involving malignant monoclonal proliferation of lymphoid cells in lymphoreticular sites, including lymph nodes, bone marrow, the spleen, the liver, and the GI tract. Presenting symptoms usually include peripheral lymphadenopathy. Compared with Hodgkin lymphoma, there is a greater likelihood of disseminated disease at the time of diagnosis. However, NHL is not one disease but rather a category of lymphocyte malignancies. Most (80 to 85%) NHLs arise from B cells, with the remainder arising from T cells or natural killer cells. However, despite the plethora of entities, treatment is often similar except in certain T-cell lymphomas.

Diffuse large B-cell lymphoma (DLBCL) is the most common subtype of non-Hodgkin lymphoma (NHL), accounting for approximately 30% of all newly diagnosed cases and more than 80% of aggressive lymphomas. Recent insights into the pathogenesis of DLBCL suggest that it is a heterogeneous group of B-cell lymphomas rather than a single clinicopathologic entity. Multiple histologic subtypes and morphologic variants are recognized, a variety of molecular and genetic abnormalities are variably present, and patients exhibit a wide range of clinical presentations and outcomes. Gene-expression profiling studies have identified at least 3 distinct molecular subtypes of DLBCL, one with an expression profile similar to normal germinal center B cells (GCB subtype), one mimicking activated peripheral-blood B cells (ABC subtype), and a third, primary mediastinal large B-cell lymphoma (PMBCL), typically presenting with mediastinal lymphadenopathy and displaying some molecular genetic similarities to Hodgkin lymphoma. A small number of cases do not fit into any of these categories and have been designated as “unclassifiable.” Although clinical indicators such as the International Prognostic Index (IPI) are used to define prognostic subgroups of DLBCL, these surrogates fail to fully reflect the underlying heterogeneity of the disease since patients with identical IPI can have strikingly different outcomes.

A consensus approach for predicting DLBCL prognosis and risk-adapted management of this lymphoma has not been achieved. In addition, the expense of gene expression profiling and the need for fresh or frozen tissue pose significant limitations on its use in routine clinical practice. Thus, there is a need in the art to develop a method to identify prognostic subgroups of DLBCL patients.

### RELEVANT LITERATURE

Natkunam et al. (2007) The oncoprotein LMO2 is expressed in normal germinal-center B cells and in human B-cell lymphomas. Blood 109:1636-1642. Natkunam et al. (2008) LMO2 protein expression predicts survival in patients with diffuse large B-cell lymphoma treated with anthracycline-based chemotherapy with and without rituximab. J. Clin Oncology 26(3):447-54. Middendorp et al. (2009) Blood. 114(11):2280-9; Furtner et al. (2005) Leukemia. 19(5):883-5; Palma et al. (2004) Int J. Cancer. 108(3):390-8; Younes et al. (2010) Am J Surg Pathol. 34(9):1266-76; Salama et al. (2009) Am J Clin Pathol. 132(1):39-49.

The original IPI was developed by Shipp et al. The International Non-Hodgkin's Lymphoma Prognostic Factors Project. A predictive model for aggressive non-Hodgkin's lymphoma. NEJM 329:987-994. (1993). A revised IPI was developed by Sehn et al. (2007) Blood 109:1857-1861, The revised International Prognostic Index (R-IPI) is a better predictor of outcome than the standard IPI for patients with diffuse large B-cell lymphoma treated with R-CHOP.

### SUMMARY OF THE INVENTION

Predictive biomarkers for Non-Hodgkin lymphoma are provided herein. Measurement of a single gene expressed by tumor cells (LMO2) and a single gene expressed by the immune microenvironment (TNFRSF9), which determination may be referred to herein as a two gene score (TGS), powerfully predicts overall survival in patients with NHL, particularly overall survival in the context of anthracycline-based chemotherapy or co-treatment with anthracycline-based chemotherapy and anti-CD20 immunotherapy. It is shown herein that increased levels of LMO2 and TNFRSF9 correlate with a positive patient response and improved prognosis. A positive response in this context is generally considered to be a progression-free survival time greater than that of a control, e.g. a placebo treated individual. The classification of a patient by the methods of the invention may be used to select a suitable therapy for the patient, to identify patient groups for clinical trials, and the like.

In some embodiments a report of this information may be provided. Such a report may then be used to predict the outcome of that person prescribed treatment with anthacycline-based chemotherapy or co-treatment of anthracycline-based chemotherapy and anti-CD20 immunotherapy, wherein a determination of increased levels of LMO2 and TNFRSF9 correlate with a positive patient response and improved prognosis.

In some embodiments of the invention, the NHL is diffuse large B cell lymphoma (DLCBCL). The assessment of LMO2 and TNFRSF9 expression may be performed by any convenient method, including detection of mRNA in a biopsy sample, detection of protein expression including without limitation, immunohistochemistry, and the like.

In some embodiments the methods of the invention integrate determination of LMO2 expression, determination of TNFRSF9 expression, and the International Prognostic Index (IPI) for NHL. While the TGS is independent of the IPI, the classification by an integrated TGS-IPI improved upon both the TGS and IPI, and thus the prognostic model incorporating both indices (TGS-IPI) provides a powerful means of segregating patients into risk groups.

In some embodiments, an expression profile of the normalized expression level of each of LMO2 and TNFRSF9. In some embodiments, the TGS is a single metric value that represents the weighted expression levels of LMO2 and TNFRSF9 in a patient sample. In some embodiments, the TGS expression representation is employed by comparing it to the TGS expression representation of one or more reference samples to arrive at a comparison result, which is then used to determine a diagnosis, a prognosis or make a prediction on responsiveness to therapy. In some embodiments, the reference sample is a cell or tissue sample with a known association with a particular risk phenotype.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. LMO2 has prognostic utility in different therapeutic eras, independently of DLBCL subtype. A) LMO2 expression as best predictor of survival within a 6-gene model in patients treated with CHOP (n=66) or R-CHOP (n=132). Univariate z-scores from Cox regression reflect magnitude of association for each gene with survival; positive and negative z-scores reflect association between higher expression of a given gene with adverse and favorable risk, respectively. Dotted lines correspond to p=0.05. (B) LMO2 over-expression is associated with GCB subtype (p<0.0001).

FIG. 2. TNFRSF9 (CD137) expression is limited to a subset of the DLBCL tumor microenvironment. A) Within sorted subpopulations, TNFRSF9 is more highly expressed in non-tumor (CD19−) than tumor cells (CD19+). B) Immunohistochemical analysis of CD137 expression among rare infiltrating cells in normal tonsil (×40, left) and a representative DLBCL from 75 tumors (×200, right). Insets show high magnification of individual positive infiltrating cells (×600). C) Flow cytometric evaluation of CD137 expression in a representative DLBCL tumor showing lack of expression among tumor cells, but detectable expression in a small proportion (5.9%) of CD4+ T-cells, and a larger proportion (28.6%) of CD8+ T-cells. D) Analysis of T-cells in tonsils, peripheral blood mononuclear cells (PBMCs), and DLBCL tumors demonstrates CD137 expression on CD4+ and CD8+ T-cells in the tumor microenvironment, but not on healthy lymphoid counterparts. E) Fraction of CD137 expressing T-cells is not correlated with total T-cell frequency in DLBCL tumors.

FIG. 3. A two gene model is independently prognostic of survival in DLBCL, and a composite model integrating IPI. A) Performance of the two gene score (TGS) comprising expression of LMO2 and TNFRSF9 as evaluated in the training (DLBCL1) cohort. Tertiles of the TGS define three risk groups with distinct Kaplan Meier estimates of survival [High Risk, TGS>−0.91; Intermediate Risk, −0.91≧TGS>−1.60; Low Risk, TGS≦−1.60]. TGS retains prognostic power within (B) GCB and (C) ABC subtypes of DLBCL treated with R-CHOP. Cases ‘unclassified’ for cell-of-origin were excluded. D) Kaplan-Meier estimates of strata using tertiles of a composite risk score integrating TGS and IPI (TGS-IPI). Depicted p-values reflect log-likelihood estimates. E) Distribution of the TGS-IPI and its relationship to survival in the training cohort, with survival modeled as a continuous function of the composite score. Tertiles of the TGS-IPI define strata depicted in (D), where means and 95% confidence intervals (CI) reflect Kaplan-Meier estimates at 2-years. Scores for individual patients are depicted as a ‘rug’ above the x-axis.

FIG. 4. External validation of the composite predictor in fixed samples by quantitative real-time polymerase chain reaction. Kaplan-Meier estimates of PFS (panels A and C), and OS (panels B and D) for strata defined by the IPI (panels A and B) or by prospectively defined thresholds of a composite model integrating the two gene score and IPI (TGS-IPI; panels C and D). The three TGS-IPI strata correspond to a priori defined thresholds depicted in FIG. 3E (High Risk, TGS-IPI>4.51; Intermediate Risk, 4.51 TGS≧3.47; Low Risk, TGS≦3.47). Depicted p-values reflect log-likelihood estimates. All depicted associations were also significant by log-rank product limit tests of Kaplan-Meier strata, including between TGS-IPI and PFS (p<0.0001, HR 3.9, 95% Cl 2.4-6.3; Panel C), and TGS-IPI and OS (p<0.0001, HR 3.7, 95% Cl 2.2-6.2; Panel D). Inset pie charts reflect distribution of risk groups as defined by the IPI or TGS-IPI, with color coding corresponding to Kaplan Meier curves.

FIG. 5. LMO2 expression has prognostic utility in different therapeutic eras, independently of DLBCL subtype. Panel A depicts univariate associations between genome-wide expression, survival, and therapeutic era. Expression profiles of 17888 genes tested for their association with OS in 414 patients treated with R-CHOP (DLBCL1, y-axis) or CHOP (DLBCL2, x-axis).^{3, 10} Each point depicts a single gene, and saturation of color within the shaded central cloud represents gene density (frequency of genes within a region); single genes occurring in low density regions are indicated by a black point. LMO2 is among the best univariate predictors considering all measured genes, in both R-CHOP and CHOP treated patients. The position of TNFRSF9 is also indicated. Genes generally tend to have significant positive correlation in prognostic influence when comparing therapeutic eras (Pearson r=0.44, p<2×10⁻¹⁶). (B) LMO2 expression is correlated (rank correlation=0.68, p<0.0001) with the Germinal Center Signature.³ (C) Receiver-operator curve (ROC) characteristics depict performance of LMO2 expression as an effective surrogate for cell of origin in independent cohorts. An optimal threshold of LMO2 expression of 1.85 (depicted by the blue-rimmed white circular marker) was derived within DLBCL1, exhibiting 87% sensitivity and 70% specificity for discrimination of GCB- from ABC-like DLBCL. The corresponding ROC profile had an area under curve of 0.84 (95% Cl 0.78-0.88, p<0.0001, binomial exact test using method of Delong et al.). This same threshold was validated in DLBCL2, where it exhibited 85% sensitivity and 73% specificity. (D) Prognostic value of LMO2 expression is independent of cell-of-origin classification, as it retains prognostic value among GCB-like DLBCL, including multivariate analyses (DLBCL1 dataset, Table 1). Patients were stratified according to LMO2 expression relative to its median level among GCB-like DLBCL. Panels E-G: LMO2 expression is a strong univariate predictor of outcome after therapy with CHOP or R-CHOP, as depicted by Kaplan Meier strata in 3 cohorts profiled by microarrays; panels E-F correspond to DLBCL1-3, respectively. Depicted strata reflect tertiles of expression of LMO2 within the corresponding cohort. For DLBCL3, tertiles were defined across all patients with DLBCL, with a subset having corresponding survival data.

FIG. 6. TNFRSF9 (CD137) expression is a strong univariate predictor of outcome after therapy with CHOP or R-CHOP, as depicted by Kaplan Meier strata in 3 cohorts profiled by microarrays. Panels A, B, and C correspond to DLBCL1, 2, and 3, respectively. Depicted strata reflect tertiles of expression of TNFRSF9 within the corresponding cohort. For DLBCL3, tertiles were defined across all patients with DLBCL, though only a subset had corresponding survival data.

FIG. 7. TNFRSF9 is highly expressed in B-cell lymphomas relative to other cancers. Variation in expression of TNFRSF9 mRNA is depicted in relation to histological classification across 1822 tumor specimens, as profiled within Expression Project for Oncology (expO) using microarrays. Vertical bars within boxes reflect median expression level, boxes depict the interquartile range, and whiskers bound 95% confidence intervals with outliers represented by single points. TNFRSF9 is more highly expressed in lymphomas than in other cancer types (p<0.0001).

FIG. 8. CD137 mRNA and protein expression levels are concordant. CCRF-CEM cells (a T-cell line) were stimulated over a 16-hour time course with ionomycin (10 ng/mL) and PMA (500 ng/mL) as previously described,¹¹ and induced CD137 expression was measured by (A) flow cytometry (FACS), (B) quantitative real-time PCR (qRT-PCR), and (C) immunohistochemistry (IHC). Enumeration of CD137 immununohistochemical staining for 10 high power fields (40×) estimated less than 5% staining among unstimulated cells, with the corresponding estimate after 16 hours of stimulation being 60%. (D) High correlation between mRNA and cell surface expression of TNFRSF9/CD137 as measured by qRT-PCR and FACS, respectively.

FIG. 9. Distribution of Two Gene Scores (TGS) and relationship to survival. Relationship of the survival model captured by TGS within the training cohort (DLBCL1), as a continuous function of the score. As a continuous variable, the bivariate model was associated with an increase in the relative risk of death of 2.7 (95% Cl, 2.0 to 3.8) per unit increment of the score. Tertiles of the TGS define the three risk strata depicted in FIG. 3A-C [i.e., High Risk, TGS>−0.91; Intermediate Risk, −0.91≧TGS>−1.60; Low Risk, TGS≦−1.60]; validity of these thresholds in other cohorts was examined in FIGS. 4 (DLBCL4), S6 (DLBCL2,3), and S9 (DLBCL4). Scores for individual patients are depicted as a ‘rug’ plot atop the x-axis.

FIG. 10. The TGS and TGS-IPI are both validated in test cohorts treated with CHOP. By applying thresholds derived from tertiles in the R-CHOP treated training cohort (DLBCL1, see Figure S5), the TGS also stratifies overall survival of patients in two test cohorts treated with CHOP (DLBCL2, panel A, p<0.0001 HR=1.8 [1.4-2.3]; and DLBCL3, panel B, p=0.0002 HR=2.0 [1.4-2.9]). Panel C captures a similar stratification for the TGS-IPI as applied to DLBCL2, p<0.0001 HR=2.0 [1.7-2.5] (IPI data were not available for DLBCL3, precluding a similar analysis).

FIG. 11. TGS is independent of the IPI. Kaplan Meier analyses reveal that TGS risk groups can further stratify individual IPI risk categories of patients treated with RCHOP (A, B, C; DLBCL1), or CHOP (D, E, F; DLBCL2), including patients with (A, D) Low (IPI=0,1), (B, E) Intermediate (IPI=2,3), or (C, F) High IPI Risk (IPI=4,5). Corresponding p-values, hazard ratios (HR), and 95% confidence intervals [95% CI] for the ternary splits of the TGS based on predefined thresholds were p=0.006 HR=3.2 (1.3-8.1) in Low IPI; p=0.03 HR=1.8 (1.0-3.0) in Intermediate IPI; and p=0.08 HR=2.1 (0.9-5.1) in High IPI groups.

FIG. 12. The TGS compares favorably to other multi-gene prognostic models. Stratification of overall survival with (A) 2 genes (using the TGS), as compared with the (B) 6-gene model of Lossos et al, and (C) the 381 genes within the Stromal Model of Lenz et al within a cohort of patients treated with R-CHOP (DLBCL1).

FIG. 13. LMO2, TNFRSF9, and the Two Gene Score (TGS) are validated in a simple assay of fixed diagnostic specimens from a cohort treated with R-CHOP. Within the external validation cohort (DLBCL4), expression of LMO2 (A, B) and TNFRSF9 (C, D) alone, and their combination as the TGS (E, F), are predictors of overall (A, C, E) and progression free survival (B, D, F). Continuous expression levels of LMO2, TNFRSF9 were individually significant as predictors of OS and PFS, and also separately within the previously defined bivariate TGS combination. p-values in OS: p=0.0003 for LMO2 [HR=0.7 (0.6-0.8)], and p=0.04 for TNFRSF9 [HR=0.8 (0.6-1.0)]; and for PFS: p<0.0001 for LMO2 [HR=0.7 (0.5-0.8)] and p=0.04 for TNFRSF9 [HR=0.8 (0.6-1.0)]. Depicted Kaplan Meier strata reflect dichotomous strata for each gene relative to its median (A-D), and the TGS relative to pre-defined ternary thresholds from DLBCL1 (E, F).

### DETAILED DESCRIPTION OF THE EMBODIMENTS

Predictive biomarkers for Non-Hodgkin lymphoma are provided herein. Measurement of a single gene expressed by tumor cells (LMO2) and a single gene expressed by the immune microenvironment (TNFRSF9) predicts overall survival in patients with NHL, particularly overall survival in the context of anthracycline-based chemotherapy or co-treatment with anthracycline-based chemotherapy and anti-CD20 immunotherapy. The classification of a patient by the methods of the invention may be used to select a suitable therapy for the patient, to identify patient groups for clinical trials, and the like.

Currently, standard regimens of anthracycline-based chemotherapy, such as R-CHOP (rituximab, cyclophosphamide, doxorubicin, vincristine, and prednisone) induce complete remission rates in DLBCL exceeding 75%. Nevertheless, current long-term event-free survival ranges from 50-60%, and 30-40% of patients eventually succumb to their disease. Predictive indices that capture such clinical heterogeneity can guide better therapeutic strategies. For instance, molecular risk assignment is useful in predicting responses to specific therapies, or to allow risk-adapted stratification within clinical trials thereby improving their statistical power. Traditional stratification schemes based on clinical characteristics such as the International Prognostic Index (IPI) have provided prognostic guidance in the management of patients with DLBCL, however the quality of prediction is improved by adding the two gene signature of the present invention.

In constructing the two gene score for LMO2 and TNFRSF9, expression values across datasets were centered and scaled to match PCR validation data. This avoids the necessity to apply corrective factors to future prospectively gathered patient sample measurements. The two-gene score (TGS) was computed in the test sets using the same weighting parameters derived in the training cohort. The thresholds for separating the training cohort into risk groups were directly applied to the validation sets. The resulting scores were tested as univariate predictors of outcome, and in multivariate combination with other variables. Within cell-of-origin subtypes, the ability of TGS to distinguish the High Risk group from the others for GCB-Like cases, and the Low Risk group from the others in ABC-Like cases in discrete fashion was tested.

To obtain a composite model integrating TGS and IPI, multivariate Cox regression was applied to the training set, with the independent variables being the previously computed TGS, together with the traditional IPI score of each patient on a scale ranging from 0 to 5. Thus, the relative weights of LMO2 and TNFRSF9 were not changed in the composite TGS-IPI model when compared with the TGS. A constant (4) was added as a term within the TGS-IPI to avoid negative scores, based on the lowest non-adjusted measurement observed across studies. The resulting scores were tested as univariate predictors of outcome, and in multivariate combination with other variables. By capturing more patients at low and high risk for progression and death, this composite index allows better risk assignment than either the IPI or TGS alone.

NHL of particular interest include DLCBCL. Among DLBCL cases assessed, all were negative for TNFRSF9 staining in tumor cells, with scattered infiltrating cells staining positively for TNFRSF9. The observation of higher TNFRSF9 expression on a minor subset of infiltrating non-neoplastic cells of DLBCL tumors reflects interactions within the local microenvironment. TNFRSF9 expression on infiltrating immune cells in DLBCL may serve as a predictive biomarker for response to a monoclonal antibody that triggers effector anti-tumor functions. Since higher expression of TNFRSF9 confers a good prognosis for DLBCL patients, such therapeutic targeting with monoclonal antibodies may provide a means of limiting chemotherapy with its attendant toxicities for this subgroup of patients.

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

International Prognostic Index (IPI). IPI is a primary clinical tool used to predict outcome for patients with aggressive NHL, based on the number of negative prognostic factors present at the time of diagnosis. Prognostic indicators include age, particularly age greater than 60 years, performance status, i.e. Eastern Cooperative Oncology Group performance status, levels of lactate dehydrogenase; number of extranodal sites of involvement, and stage of disease. See, for example, Sehn et al., supra. and Shipp et al., supra., each herein specifically incorporated by reference.

“LMO2′, “LIM Domain Only 2”, “LMO2 protein”, LIM Doman Only 2 protein”, LMO2 polypeptide” and LIM Domain Only 2 polypeptide” are used interchangeably herein to refer to a polypeptide that belongs to the LMO class of cysteine-rich peptides containing a LIM motif and that function as modulators of transcription. The gene LMO2 encodes a protein of 158 amino acids, described at, for example, Genbank NM_{—}005574. LMO2 polypeptide plays a critical role in hematopoietic cell development. It is expressed in many normal hematopoietic populations, including germinal center B-cells, myeloid precursors, erythroid precursors and megakaryocytes, as well as in abnormal hematopoietic populations such as B cell lymphomas, lymphoblastic leukemia and acute myeloid leukemia.

An “anti-LMO2 antibody” suitable for use in the methods described herein can be any monoclonal or polyclonal antibody with specificity for the polypeptide LMO2. Antibodies with specificity for LMO2 can be prepared by methods that are well understood in the art. Preferred antibody compositions are ones that have been selected for antibodies directed against a polypeptide or polypeptides of LMO2. Particularly preferred polyclonal antibody preparations are ones that contain only antibodies directed against a polypeptide or polypeptides of LMO2. In one preferred embodiment, the anti-LMO2 antibody is the monoclonal antibody 1A9-1 (Natkunam et al. Blood. 2007 Feb. 15; 109(4):1636-42. Epub 2006 Oct. 12.).

TNFRSF9. TNFRSF9 is a member of the tumor necrosis factor (TNF) receptor family. Members of this receptor family and their structurally related ligands are important regulators of a wide variety of physiologic processes and play an important role in the regulation of immune responses. The protein may also be referred to as ILA (induced by lymphocyte activation); 4-1BB, CD137 or Ly63.

The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The human protein binds only to its cognate ligand, TNFSF9. The genetic sequence of the gene, encoded protein and cDNA for human TNFRSF9 may be found at Genbank, accession number AY438976, herein specifically incorporated by reference. The protein identification number is AAR05440.1.

“Anthracycline based chemotherapy” as used herein refers to therapeutics that comprise the anthracycline class of antibiotic compounds. Anthracyclines are used to treat a wide range of cancers, including leukemias, lymphomas, and breast, uterine, ovarian and lung cancers. Examples of anthracyclines include daunorubicin, doxorubicin, epirubicin, idarubicin and valrubicin. An example of an anthracycline-based therapy would be one comprising cyclophosphamide, vincristine, doxorubicin and prednisone (CHOP). An example of an anthracycline based-therapy provided in conjunction with an anti-CD20 immunotherapy would be one comprising CHOP and the anti-CD20 antibody rituximab, i.e. R-CHOP.

“Anti-CD20 immunotherapy” as used herein refers to therapeutics comprised of anti-CD20 antibodies, which are defined in further detail below. Such therapeutics have a demonstrated utility for the treatment of B cell lymphomas and leukemias.

The terms “antibody” and “antibody substance” as used interchangeably herein refer to immunoglobulin molecules and immunologically active portions of immunoglobulin molecules, i.e., molecules that contain an antigen binding site which specifically binds an antigen. An antibody which specifically binds to a given polypeptide of an antigen is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides monoclonal and polyclonal antibodies.

The term “monoclonal antibody” or “monoclonal antibody composition” as used herein refers to a population of antibody molecules that contain only one species of an antigen binding site capable of immunoreacting with a particular epitope of an antigen. The term “polyclonal antibodies” or “polyclonal antibody composition” refers to a population of antibody molecules that contain multiple species of antibodies capable of reacting with different epitopes of the same antigen.

The term “anti-CD20 antibody” refers herein to monoclonal or polyclonal antibodies with specificity for the polypeptide CD20. Antibodies with specificity for CD20 can be prepared by methods that are well understood in the art. Preferred antibody compositions are ones that have been selected for antibodies directed against a polypeptide or polypeptides of CD20. Particularly preferred polyclonal antibody preparations are ones that contain only antibodies directed against a polypeptide or polypeptides of CD20. Anti-CD20 antibodies that are suitable for use in the current invention would include, for example, rituximab, ibritumomab tiuxetan, tositumomab, AME-133v (Applied Molecular Evolution), Ocrelizumab (Roche), Ofatumumab (Genmab), TRU-015 (Trubion) and IMMU-106 (Immunomedics). In one embodiment, the anti-CD 20 antibody is rituximab.

“CD20”, “CD20 protein”, and “CD20 polypeptide” are used interchangeably herein to refer to a polypeptide encoded by a member of the membrane-spanning 4A gene family. This gene, referred to as “MS4A1”, “Membrane-spanning 4-domains, subfamily A, member 1”, “B1” and “B-lymphocyte surface antigen B1”, encodes a non-glycosylated phosphoprotein of 297 amino acids, as described at, for example, Genbank NM_{—}152866 and Genbank NM_{—}021950 (alternative splice variants that encode the same protein). CD20 polypeptide is expressed on the surface of B cells beginning at the late pre-B cell phase of development, and plays a role in B cell proliferation.

Inducible costimulatory molecule. As used herein, an inducible costimulatory molecule is a polypeptide expressed on immune cells, including without limitation natural killer (NK) cells, which expression is induced or significantly upregulated during activation of NK cells. Activation of the costimulatory molecule enhanced the effector cell function, for example increasing ADCC mediated by the activated NK cells. Such inducible costimulatory molecules are known to those of skill in the art, and include, without limitation, CD137, OX40, GITR, CD30, ICOS, etc. Agonists of such molecules, including antibodies that bind to and activate the costimulatory molecule, are of interest for the methods of the invention. Many such costimulatory molecules are members of the tumor necroses factor receptor family (TNFR). TNFR-related molecules do not have any known enzymatic activity and depend on the recruitment of cytoplasmic proteins for the activation of downstream signaling pathways.

“Inducible costimulatory molecule agonist” includes the native ligands, as described above, aptamers, antibodies specific for an inducible costimulatory molecule that activate the receptor, and derivatives, variants, and biologically active fragments of antibodies that selectively bind to an inducible costimulatory molecule. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The variant polypeptides can be naturally or non-naturally glycosylated, i.e., the polypeptide has a glycosylation pattern that differs from the glycosylation pattern found in the corresponding naturally occurring protein.

Fragments of the ligand or antibodies specific for an inducible costimulatory molecule, particularly biologically active fragments and/or fragments corresponding to functional domains, are of interest. Fragments of interest will typically be at least about 10 aa to at least about 15 aa in length, usually at least about 50 aa in length, but will usually not exceed about 200 aa in length, where the fragment will have a contiguous stretch of amino acids that is identical to the polypeptide from which it is derived. A fragment “at least 20 aa in length,” for example, is intended to include 20 or more contiguous amino acids from, for example, an antibody specific for CD137, or from TNFSF9. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods. A “fusion” polypeptide is a polypeptide comprising a polypeptide or portion (e.g., one or more domains) thereof fused or bonded to heterologous polypeptide.

In some embodiments, the inducible costimulatory molecule agonist is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogeneic human antibodies, which produce less of an immune response when administered to humans, are preferred for use in the present invention.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ricin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

By “enhancing efficacy” is meant an increase in ADCC-mediated apoptosis of tumor cells compared to level of apoptosis observed with a single agent, e.g. a monoclonal antibody specific for a tumor cell. By synergistic, it is meant that a combination of agents provides for an effect greater than a single agent, which effect may be greater than the additive effect of the combined agents.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a person with cancer is “responsive” to a treatment if at least one symptom of the cancer is alleviated, terminated, slowed, or prevented. As used herein, a person with cancer is also “responsive” to a treatment if recurrence or metastasis of the cancer is reduced, slowed, delayed or prevented.

A biopsy sample suitable for use in the methods described herein is one that is collected from a lymphoma from a person with B-cell lymphoma. A lymphoma is a solid neoplasm of lymphocyte origin, and is most often found in the lymphoid tissue. Thus, for example, a biopsy from a lymph node, e.g. a tonsil, containing such a lymphoma would constitute a suitable biopsy. In one embodiment, the B cell lymphoma is a diffuse large B cell lymphoma.

“Immunohistochemistry” as used herein refers to the technique of visualizing the presence of a polypeptide in a cell in a tissue with an antibody that is specific for the polypeptide. Generally, the tissue is fixed, thinly sliced, and incubated with the antibody, during which time the antibody will hybridize to the target polypeptide. Unbound antibody is washed away and the bound antibody is visualized, either directly or indirectly, by microscopy.

Before the present active agents and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol NO. I, Cold Spring Harbor Laboratory (1998); and Harlow and Lane, Antibodies: A Laboratory Manual, Cold Spring Harbor Laboratory; (1988).

### DESCRIPTION OF THE METHOD

Non-Hodgkin lymphomas are a heterogeneous group of disorders involving malignant monoclonal proliferation of lymphoid cells in lymphoreticular sites, including lymph nodes, bone marrow, the spleen, the liver, and the GI tract. Presenting symptoms usually include peripheral lymphadenopathy. Compared with Hodgkin lymphoma, there is a greater likelihood of disseminated disease at the time of diagnosis. Diagnosis is usually based on lymph node or bone marrow biopsy or both. Conventional treatment involves radiation therapy, chemotherapy, or both.

Most (80 to 85%) NHLs arise from B cells; the remainder arise from T cells or natural killer cells. Either precursor or mature cells may be involved. Overlap exists between leukemia and NHL because both involve proliferation of lymphocytes or their precursors. A leukemia-like picture with peripheral lymphocytosis and bone marrow involvement may be present in up to 50% of children and in about 20% of adults with some types of NHL. A prominent leukemic phase is less common in aggressive lymphomas, except Burkitt's and lymphoblastic lymphomas.

Specific diseases include, without limitation, precursor B-lymphoblastic leukemia/lymphoma; B-cell chronic lymphocytic leukemia/small lymphocytic lymphoma; B-cell prolymphocytic leukemia; lymphoplasmacytic lymphoma; splenic marginal zone B-cell lymphoma (±villous lymphocytes); hairy cell leukemia; plasma cell myeloma/plasmacytomas; extranodal marginal zone B-cell lymphoma of the MALT type; nodal marginal zone B-cell lymphoma (±monocytoid B cells); follicular lymphoma; mantle cell lymphoma; diffuse large B-cell lymphomas (including mediastinal large B-cell lymphoma and primary effusion lymphoma); and Burkitt's lymphoma, and particularly DLCBCL.

In the method, a biopsy of a lymphoma is collected from the person with B-cell lymphoma. The biopsy may be of any lymphoma in the person, for example, a lymphoma in a lymph node. In one embodiment, the B-cell lymphoma is a diffuse large B cell lymphoma. The biopsy is assessed to determine the expression of LMO2 and TNFRSF9. Assessment can be by any of a number of methods understood in the art for assessing the expression of a gene in a cell.

In practicing the subject methods, a two gene score (TGS) is obtained for a biopsy sample from a patient, representing the expression levels of LMO2 and TNFRSF9. Increased expression of these two markers is indicative of a positive prognosis. The analysis may be integrated with an IPI score to provide for increased prognostic ability. To obtain a TGS, the expression level of the two genes is first measured/determined.

Expression levels are obtained by analysis of a tumor biopsy sample from a subject. A sample that is collected may be freshly assayed or it may be stored and assayed at a later time. If the latter, the sample may be stored by any means known in the art to be appropriate in view of the method chosen for assaying gene expression, discussed further below. For example the sample may freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis. In some instances, as when the sample is a tissue biopsy that will be sectioned for analysis, the sample may be embedded in sectioning medium, e.g. OCT or paraffin. The sample is then sectioned, and one or more sections are then assayed to measure the expression levels of the two genes.

The expression levels of the genes may be measured by polynucleotide, i.e. mRNA, levels or at protein levels. Exemplary methods known in the art for measuring mRNA expression levels in a sample include hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), PCR-based methods (e.g. reverse transcription PCR(RT-PCR) (Weis et al., Trends in Genetics 8:263-264 (1992)), and antibody-based methods, e.g. immunoassays, e.g., enzyme-linked immunosorbent assays (ELISAs), immunohistochemistry, and flow cytometry (FACS).

For measuring mRNA levels, the starting material is typically total RNA or poly A+ RNA isolated from a suspension of cells, e.g. a peripheral blood sample a bone marrow sample, etc., or from a homogenized tissue, e.g. a homogenized biopsy sample, a homogenized paraffin- or OCT-embedded sample, etc. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g. as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

For measuring protein levels, the amount or level of one or more proteins/polypeptides in the sample is determined, e.g., the protein/polypeptide encoded by the gene of interest. In such cases, any convenient protocol for evaluating protein levels may be employed wherein the level of one or more proteins in the assayed sample is determined.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 250-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAPTM microsphere technology, western blotting, immunohistochemistry, and flow cytometry. In, for example, flow cytometry methods, the quantitative level of gene products of one or more TGS genes are detected on cells in a cell suspension by lasers. As with ELISAs and immunohistochemistry, antibodies (e.g., monoclonal antibodies) that specifically bind the TGS polypeptides are used in such methods.

The resultant data provides information regarding expression for each of the genes that have been probed, wherein the expression information is in terms of whether or not the gene is expressed and, typically, at what level, and wherein the expression data may be both qualitative and quantitative.

Once the expression level of the genes has been determined, the measurement(s) may be analyzed in any of a number of ways to obtain an expression representation. For example, an expression profile may be the normalized expression level of the genes in a patient sample. An expression profile may be generated by any of a number of methods known in the art. For example, the expression level of each gene may be log₂ transformed and normalized relative to the expression of a selected housekeeping gene, e.g. ABL1, GAPDH, or PGK1, or relative to the signal across a whole microarray, etc.

The relative expression levels may be provided as a signature that is a single metric value that represents the weighted expression levels of the genes assayed in a patient sample, where the weighted expression levels are defined by the dataset from which the patient sample was obtained. A signature for a patient sample may be calculated by any of a number of methods known in the art for calculating gene signatures. For example, the expression levels of each of the genes in a patient sample may be log₂ transformed and normalized. The normalized expression levels for each gene is then weighted by multiplying the normalized level to a weighting factor, or “weight”, to arrive at weighted expression levels for each of the one or more genes. The weighted expression levels are then totaled and in some cases averaged to arrive at a single weighted expression level for the genes analyzed. The weighting factor, or weight, is usually determined by Principle Component Analysis (PCA) of the dataset from which the sample was obtained.

As a preferred example, the expression representation is provided as a two gene score (TGS), which is a single metric value that represents the sum of the weighted expression levels of the two genes in a patient sample. A TGS is determined by methods very similar to those described above for a signature, e.g. the expression levels of each of the two genes in a patient sample may be log₂ transformed and normalized; the normalized expression levels for each gene is then weighted by multiplying the normalized level to a weighting factor, or “weight”, to arrive at weighted expression levels for each of the one or more genes; and the weighted expression levels are then totaled and in some cases averaged to arrive at a single weighted expression level for the genes analyzed. However, in contrast to a signature, the weighted expression levels are defined by a reference dataset, or “training dataset”, e.g. by Principle Component Analysis of a reference dataset. Any dataset relating to patients having hematological malignancies may be used as a reference dataset. For example, the weights may be determined based upon any of the datasets provided in the examples section below.

This analysis may be readily performed by one of ordinary skill in the art by employing a computer-based system, e.g. using any hardware, software and data storage medium as is known in the art, and employing any algorithms convenient for such analysis.

Evaluating a subject. The gene expression information of the invention may employed to provide a prognosis to a patient with a hematological malignancy, and/or to provide a prediction of the responsiveness of a patient to a therapy. Typically, expression information is employed by comparison to a reference or control, and using the results of that comparison (a “comparison result”) to determine a prognosis or prediction. The terms “reference” and “control” as used herein mean a standardized gene expression representation to be used to interpret the analysis of a given patient and assign a prognostic, and/or responsiveness class thereto. The reference or control is typically a TGS that is obtained from a cell/tissue with a known diagnosis.

In certain embodiments, an obtained TGS is compared to a single reference/control TGS, in other embodiments, the obtained TGS is compared to two or more different reference/control TGS. For example, a TGS may be compared to both a positive TGS and a negative TGS to obtain confirmed information regarding whether the tissue has the prognosis of interest. Patients can be ascribed to high- or low-risk categories, or high-, intermediate- or low-risk categories, for overall survival, relapse-free survival, event-free survival, etc. depending on whether their expression representation is higher or lower than the median across a cohort of patients with the same disease.

Alternatively or additionally, the expression representation may be employed to provide a prediction of responsiveness of a patient to a particular therapy. These predictive methods can be used to assist patients and physicians in making treatment decisions, e.g. in choosing the most appropriate treatment modalities for any particular patient. For example, the expression representation may be used to predict responsiveness to chemotherapy and to combinations of chemotherapy; to antibody therapy, e.g. anti-CD137, anti-CD20, etc., or to stem cell transplantation, e.g. allogenic hematopoietic stem cell transplantation, e.g. from bone marrow. Additionally, the expression representation may be used on samples collected from patients in a clinical trial and the results of the test used in conjunction with patient outcomes in order to determine whether subgroups of patients are more or less likely to show a response to a new drug than the whole group or other subgroups. Further, such methods can be used to identify from clinical data the subsets of patients who can benefit from therapy. Additionally, a patient is more likely to be included in a clinical trial if the results of the test indicate a higher likelihood that the patient will have a poor clinical outcome if treated with more standardized treatments, and a patient is less likely to be included in a clinical trial if the results of the test indicate a lower likelihood that the patient will have a poor clinical outcome if treated with more standardized treatments.

The subject methods can be used alone or in combination with other clinical methods for patient stratification known in the art, particularly the IPI, to provide a diagnosis, a prognosis, or a prediction of responsiveness to therapy.

In some embodiments, providing an evaluation of a subject includes generating a written report that includes the artisan's assessment of the subject's current state of health, of the subject's prognosis, i.e. a “prognosis assessment”, and/or of possible treatment regimens, i.e. a “treatment assessment”. Thus, a subject method may further include a step of generating or outputting a report providing the results of a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a prognosis assessment, and/or a treatment assessment and its results. A subject report can be completely or partially electronically generated. A subject report includes at least a prognosis assessment, i.e. a prediction of the likelihood that a patient with a cancer will have a cancer-attributable death or progression, including recurrence, metastatic spread, and drug resistance; or a treatment assessment, i.e. a prediction as to the likelihood that a cancer patient will have a particular clinical response to treatment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include the gene expression levels of the genes, and/or a TGS signature and/or TGS score, b) reference values employed, if any; 6) other features.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will have a cancer-attributable death or progression. The interpretive report can include, for example, results of the gene expression analysis, methods used to calculate the TGS expression representation, and interpretation, i.e. prognosis. The assessment portion of the report can optionally also include a Recommendation(s). For example, where the results indicate that the subject will be responsive to induction chemotherapy, the recommendation can include a recommendation that a bone marrow transplant be performed with induction chemotherapy to follow.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Kits

The detection reagents can be provided as part of a kit. Thus, the invention further provides kits for measuring LMO2 and TNFRSF9 in a biological sample. Procedures using these kits can be performed by clinical laboratories, experimental laboratories, medical practitioners, or private individuals. The kits of the invention may comprise amplification and/or sequencing primers, and/or hybridization primers or antibodies for protein determination. The kit may optionally provide additional components that are useful in the procedure, including, but not limited to, buffers, developing reagents, labels, reacting surfaces, means for detection, control samples, standards, instructions, and interpretive information.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that may be present is a website address which may be used via the internet to access the information at a removed site. Any convenient means may be present in the kits.

In addition to instructions for using the components of the kit, the kit can further include instructions of analyzing the data acquired from the assays described herein. For example, the instructions can include a graph and/or table of known statistics for the probabilities of overall survival and progression free survival following anthracycline-based chemotherapy or anthracycline-based chemotherapy in conjunction with anti-CD20 immunotherapy for persons with lymphomas having differing expression of the genes of the invention. In addition, instructions can be provided to interpret these graphs and/or tables. These graphs and/or tables and instructions would be generally recorded on a suitable recording medium, for example, printed on a substrate such as paper or plastic. Alternatively, these graphs and/or tables and instructions can be provided on an electronic storage data file present on a suitable computer readable storage medium, e.g. CD-ROM, diskette, etc. In some embodiments, the actual graphs and/or table and instructions are not present in the kit, but means for obtaining the graphs/tables and instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXPERIMENTAL

The following example is provided to further illustrate the advantages and features of the present invention, but is not intended to limit the scope of the invention. While they are typical of those that might be used, other procedures, methodologies, or techniques known to those skilled in the art may alternatively be used.

### Example 1

### Prediction of Survival in Diffuse Large B-Cell Lymphoma Based On the Expression of Two Genes Reflecting Tumor and Microenvironment

Several gene expression signatures predict survival in diffuse large B cell lymphoma (DLBCL), but the lack of practical methods for genome scale analysis has limited translation to clinical practice. We examined the power of individual genes to predict survival across different therapeutic eras. In studying 787 patients with DLBCL, we built and validated a simple model employing one gene expressed by tumor cells and another expressed by host immune cells, assessing added prognostic value to the clinical International Prognostic Index (IPI). We validated models in an independent cohort using diagnostic formalin-fixed specimens. We verified expression of LMO2 as an independent predictor of survival and ‘Germinal Center B-cell’ subtype. We identified expression of TNFRSF9 from the tumor microenvironment, as the best in bivariate combination with LMO2. We studied distribution of TNFRSF9 tissue expression in 95 patients. A model integrating these two genes was independent of ‘cell of origin’ classification, ‘stromal signatures’, IPI, and added to the predictive power of the IPI. This bivariate model and a composite score integrating the IPI performed well in three independent cohorts of 545 previously described patients. Both models robustly stratified outcomes in a simple assay of routine specimens from 147 newly diagnosed patients.

Measurement of a single gene expressed by tumor cells (LMO2) and a single gene expressed by the immune microenvironment (TNFRSF9) powerfully predicts overall survival in patients with DLBCL. A simple test integrating these two genes with the IPI can readily be used to select patients of different risk groups for clinical trials.

### Introduction

The most common subtype of non-Hodgkin lymphoma—diffuse large B-cell lymphoma (DLBCL)—is clinically heterogeneous. Currently, standard regimens such as R-CHOP (rituximab, cyclophosphamide, doxorubicin, vincristine, and prednisone) induce complete remission rates in DLBCL exceeding 75%. Nevertheless, current long-term event-free survival ranges from 50-60%, and 30-40% of patients eventually succumb to their disease. Predictive indices that capture such clinical heterogeneity might guide better therapeutic strategies. For instance, molecular risk assignment could be used to predict responses to specific therapies, or to allow risk-adapted stratification within clinical trials thereby improving their statistical power. Traditional stratification schemes based on clinical characteristics such as the International Prognostic Index (IPI) have provided prognostic guidance in the management of patients with DLBCL. Despite their ease of implementation, clinical prognostic indices have not yet translated into risk-adapted therapeutic strategies for DLBCL. One reason for this limitation is that IPI does not fully represent disease heterogeneity. Therefore, efforts have shifted to broad molecular profiles that model and stratify risks of adverse outcomes.

Using genome-scale expression profiles, we previously defined two distinct subtypes of DLBCL with different normal counterparts and clinical outcomes. With their distinct biological and clinical features independently validated, these two DLBCL groupings—Germinal Center B-cell like (GCB-like) and Activated B-cell like (ABC-like)—are recognized as DLBCL subtypes in the current World Health Organization classification. However, the current lack of standardized methods for routine clinical use of expression profiles and requirement for fresh or frozen tissues has limited their clinical utility.

Therefore, to develop a practical clinical risk tool we examined associations between genome-wide expression profiles and outcomes at the single gene level. We aimed to construct prognostic models that integrate clinical and molecular indices in the current therapeutic era, then to test and validate them in a simple assay amenable to routine clinical practice.

### Methods

STUDY COHORTS: Institutional Review Board (IRB) approval was obtained from all participating institutions for inclusion of coded and de-identified patient data in this study. Gene expression and clinical data were analyzed for 787 adult patients with DLBCL including 2 cohorts treated with R-CHOP (DLBCL1 and 4) and 2 with CHOP (DLBCL 2 and 3), as detailed in Table 1. These included 3 previously described multinational cohorts comprising 545 patients whose frozen tumors were profiled using microarrays (i.e., DLBCL1-3), and an independent cohort of 147 patients whose fixed, paraffin embedded tumors were profiled using quantitative real-time polymerase chain reaction (DLBCL4). An additional 95 patients were assessed for cell surface expression of CD137 by flow cytometry or immunohistochemistry, with biopsies obtained at Stanford University or the Norwegian Radium Hospital, Oslo, Norway. Normal tonsils were obtained from routine tonsillectomies of 4 pediatric patients at Lucille Packard Children's Hospital, and peripheral blood of 22 healthy donors was used as a source of mononuclear cells.

For external validation of models, we tested them in an independent cohort of 147 patients treated with R-CHOP (DLBCL4), using diagnostic specimens from patients treated at the British Columbia Cancer Agency (n=64), Stanford University Medical Center (n=49), University of Miami Cancer Center (n=23), and Hospital Santa Creu i Sant Pau in Barcelona (n=11). Cases were selected based on: (1) diagnosis of de novo DLBCL of any clinical stage; (2) availability of tissue obtained at diagnosis prior to therapy; (3) treatment with curative intent with R-CHOP (DLBCL1 and DLBCL4) or CHOP (DLBCL2 and DLBCL3); and (4) availability of outcome data at the treating institution. Criteria commonly used for prospective trial enrollment such as normal renal and liver functions, absence of co-morbid conditions, and good performance status were not applied for case selection. Patients with primary mediastinal large B-cell lymphoma or central nervous system involvement at presentation were excluded. Follow-up information was obtained from the patients' medical records and included response to initial therapy based on the Cheson criteria, and the clinical endpoints analyzed included overall (OS), and progression free (PFS), with data censored for patients who did not have an event at the last follow-up visit. Histological sections were reviewed to confirm the diagnoses based on features of DLBCL according to the World Health Organization classification.

STATISTICAL METHODS: Univariate associations between expression profiles and survival were assessed by Cox regression using the coxph function from the R statistical software package. Differences between survival curves were assessed by the Kaplan-Meier product limit method. Statistical significance was measured using the log-likelihood statistic for continuous association between expression and outcome, and the log-rank method for discretely stratified patient groupings as previously described. Data from DLBCL1 were used to build survival models, with DLBCL2-4 cohorts used exclusively for validation. Bivariate combinations of genes with LMO2 were tested for their ability to predict survival using multivariate Cox regression.

In constructing the final model comprising LMO2 and TNFRSF9, expression values across the microarray datasets were centered and scaled to match PCR validation data (detailed below). This avoids the necessity to apply corrective factors to future prospectively gathered patient sample measurements. The two-gene score (TGS) was computed in the test sets (DLBCL2, 3, and 4) using the same weighting parameters derived in the training cohort (DLBCL1). The thresholds for separating the training cohort into risk groups were directly applied to the validation sets. The resulting scores were tested as univariate predictors of outcome, and in multivariate combination with other variables. Within cell-of-origin subtypes, we tested for ability of TGS to distinguish the High Risk group from the others for GCB-Like cases, and the Low Risk group from the others in ABC-Like cases in discrete fashion (log-rank test), as well as testing TGS as a continuous variable (Cox log-likelihood test). Continuous risk score curves were generated using the coxph function in the R software package, estimating the baseline using the Breslow method. Resulting confidence intervals for survival at each time were smoothed using cubic splines.

To obtain a composite model (i.e., the TGS-IPI) incorporating LMO2, TNFRSF9, and IPI, we applied multivariate Cox regression to the training set, with the independent variables being the previously computed TGS, together with the traditional IPI score of each patient (on a scale ranging from 0 to 5). Thus, the relative weights of LMO2 and TNFRSF9 were not changed in the composite TGS-IPI model when compared with the TGS. A constant (4) was added as a term within the TGS-IPI to avoid negative scores, based on the lowest non-adjusted measurement observed across studies. For a minority of patients in the training cohort, evaluation of all IPI risk factors was not available, where it invariably related to patients missing only one of the five component risk factors. In those cases, we did not employ imputation and instead used the average of the minimum and maximum possible IPI score defined by available factors. The validation cohort had no missing data for components of the TGS, IPI, OS, or PFS. As with the TGS, the TGS-IPI was also computed in the test sets using the same parameters derived in the training cohort (DLBCL1). The resulting scores were tested as univariate predictors of outcome, and in multivariate combination with other variables.

Alternate versions of the TGS with equal weightings of LMO2 and TNFRSF9 (i.e., TGS=−LMO2−TNFRSF9) and the TGS-IPI (TGS-IPI=2×IPI−LMO2−TNFRSF9) yielded nearly identical results within test and validation cohorts, suggesting that the simplicity of these equations might promote their clinical adoption in the manner of the IPI. However, we opted to keep the weightings in the final models of TGS and TGS-IPI, since the quantitative measurements of the two genes are likely to require arithmetic that is unlikely to be done at the bedside. To facilitate calculation of the risk scores and corresponding risk groups based on the described thresholds for the TGS, IPI, and TGS-IPI, we devised an online calculator.

IMMUNOHISTOCHEMISTRY: Serial sections of 4 microns were cut from formalin fixed, paraffin embedded specimens and deparaffinized in xylene and hydrated in a series of graded alcohols. A mouse monoclonal anti-CD137 (clone BBK-2, Neomarkers, Fremont, Calif.) was used at a dilution of 1:15. Antigen retrieval by microwave pretreatment was performed in citric acid buffer (10 mM, pH 6.0, for 10 minutes). Detection was performed using the EnVision system (Dako, Carpinteria, Calif.). Among 75 DLBCL cases assessed, all were negative for CD137 staining in tumor cells, with scattered infiltrating cells staining positively for CD137.

FLOW CYTOMETRY: Tumor specimens were obtained at diagnosis and single cell suspensions were prepared and frozen as described previously. Tonsils were similarly handled. PBMC from healthy individuals (n=22) were isolated using density gradient separation by Ficoll-Paque Plus (Amersham Biosciences, Uppsala, Sweden). Monoclonal antibodies used to stain human primary lymphoma specimens (n=20), tonsils (n=4), and PBMCs (n=22) included: CD4 Pacific Blue, CD8 FITC, CD20 APC-Cy7, CD25 PE and CD45RO PE-Cy7 (all from Becton Dickinson Biosciences (BD), CA), CD3 QD605 (Invitrogen), and CD137 APC (clone 4B4-1, Biosource). Stained cells were detected on a FACSCalibur or LSR11 three laser cytometer (BD) and analyzed using Cytobank.

MICROARRAY DATA: Gene expression data for 414 adult patients with DLBCL, treated with R-CHOP (n=233; DLBCL1) or CHOP (n=181; DLBCL2), and profiled with Affymetrix HG-U133 Plus 2.0 microarrays by Lenz et al and the LLMPP, were obtained from the NCBI Gene Expression Omnibus (GSE10846).

A set of 131 patients with DLBCL and treated primarily with CHOP-based regimen and profiled with Affymetrix HG-U133A microarrays by Hummel et al. was used as an additional test cohort (DLBCL3) for the TGS (IPI was not available); expression data were obtained from NCBI GEO (GSE4475). Within DLBCL3, we excluded Burkitt Lymphoma (BL) cases by selecting only cases that had p<0.05 for predicted molecular classification as BL as defined by the authors, leaving 131 patients with DLBCL among whom 96 had survival data.

We used two tailed t-tests and analysis of variance for the estimation of significant differences in gene expression level across cancer types profiled as part of the Expression Project for Oncology (expO), obtained from NCBI GEO as GSE2109.

For DLBCL expression profiles, probe set summaries were derived from Affymetrix microarray data from raw CEL files using a custom Chip Definition File (CDF) mapping to Entrez Gene identifiers (version 12). Data were normalized with MASS, with intensities scaled to a global median of 500 for each array. Base-2 logarithms of normalized measurements were employed in estimating mRNA expression levels for LMO2 and TNFRSF9, and for corresponding statistical measures for associations with cell-of-origin, International Prognostic Index, and outcomes including OS.

RNA ISOLATION & QUANTITATIVE REAL-TIME PCR: For DLBCL4, using diagnostic fixed archival specimens, total RNA was extracted from two 5-μm-thick slices of formalin-fixed, paraffin-embedded tissue sections, as previously reported. Total RNA (2 μg) was reverse transcribed using a High-Capacity cDNA Reverse Transcription Kit (Applied Biosystems, Foster City, Calif.).⁶ Real-time reverse transcription-PCR was performed using an ABI PRISM 7900HT (Applied Biosystems), as previously reported. Briefly, commercially available Assays-on-Demand, consisting of a mix of unlabeled PCR primers and TaqMan minor groove binder probe (FAM dye-labeled) were used for measurement of expression of LMO2 (Hs00277106 ml), and TNFRSF9 (Hs00155512 ml). For endogenous control, we used phosphoglycerate kinase 1 (PGK1) with VIC-dye labeled Pre-Developed Assay Reagent (Applied Biosystems). PCR reactions were prepared in a final volume of 20 μL, with final concentrations of 1× TaqMan Universal PCR Master Mix (Applied Biosystems) and cDNA derived from 20 ng input RNA. Thermal cycling conditions included an initial uracil-N-glycosylase (UNG) incubation at 50° C. for 2 minutes, AmpliTaq Gold DNA Polymerase activation at 95° C. for 10 minutes, 40 cycles of denaturation at 95° C. for 15 seconds, and annealing and extension at 60° C. for 1 minute. Each measurement was performed in triplicate. The fractional cycle number at which the amount of amplified target reached a fixed threshold (Ct) was determined as previously reported. TNFRSF9 and LMO2 mRNA levels were normalized to PGK1 expression and calculated by the delta-CT method. For calibration, we used cDNA from the Raji lymphoma cell line (ATCC) obtaining delta-delta-Ct values for TNFRSF9 and LMO2 in each sample.

### Results

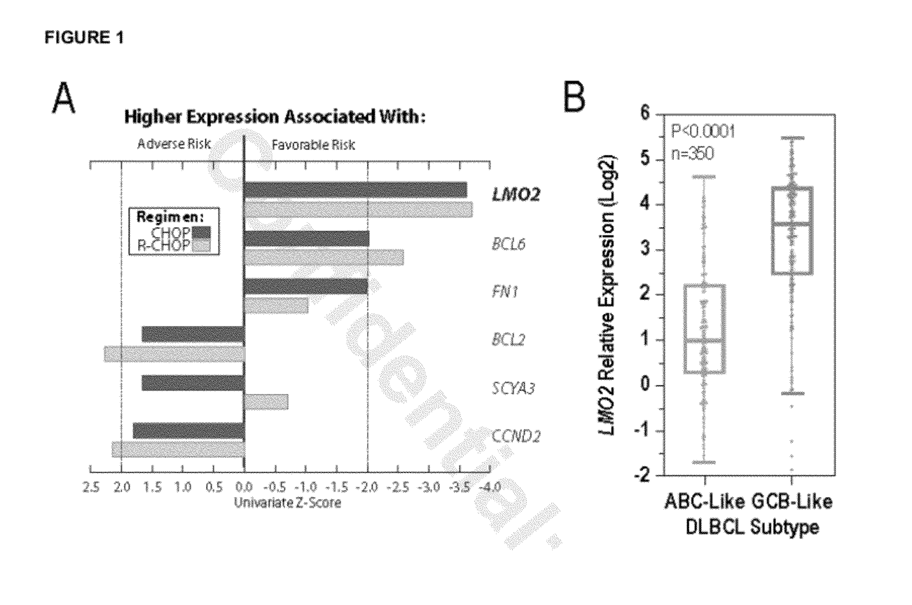

LMO2 EXPRESSION IS A ROBUST PROGNOSTIC DETERMINANT IN DLBCL. Among a previously described set of 6 genes whose expression is predictive of survival of patients with DLBCL independently of measurement platform, or use of frozen or fixed biopsy specimens, LMO2 was the single gene with strongest independent prognostic value in distinct therapeutic eras (FIG. 1A). By reevaluating univariate associations between overall survival (OS) and expression of nearly the entire transcriptome, LMO2 again emerged as exceptionally prognostic in publicly available data from 414 previously described patients treated by either CHOP or R-CHOP (Table 1).

As exemplified by LMO2, the prognostic power of most genes tended to be significantly correlated in patients treated with CHOP or with R-CHOP (p<0.0001, Figure S1A). In this cross-therapy analysis, expression of LMO2 was the top ranking survival predictor among genes distinguishing tumor cell of origin (FIGS. 1B and S1A-C). Indeed, LMO2 expression was significantly higher among the GCB-like subtype (p<0.0001, FIG. 1B), was strongly correlated with expression of the “germinal center signature” (p<0.0001, Figure S1B), and was effective as a diagnostic test for capturing tumor cell of origin (sensitivity 85%, specificity 73%, Figure S1C). Nonetheless, LMO2 expression added to cell of origin classification in stratifying outcomes in multivariate analyses, carrying prognostic value even among GCB-like DLBCL (Table S1 and Figure S1D). We therefore verified LMO2 expression as an exceptional predictor of both cell-of-origin classification and of survival in distinct therapeutic eras (Figure S1E-G), providing independent prognostic value irrespective of measurement methodology.

TNFRSF9 EXPRESSION IS INDEPENDENTLY PROGNOSTIC. Prior studies demonstrated significant prognostic influence derived from stromal signatures in diverse lymphomas. We aimed to capture this contribution by constructing a bivariate survival predictor, evaluating pair-wise models including LMO2 along with a second gene more highly expressed in non-tumor cells. We assessed contribution from the microenvironment by comparing expression of paired sorted tumor (CD19+) and non-tumor (CD19−) fractions from DLBCL tumors. Among genes more highly expressed in non-tumor cells, TNFRSF9, encoding CD137 (also known as 4-1 BB), was the best in bivariate combination with LMO2 (Table S2). Higher TNFRSF9 was also a strong univariate predictor of good outcomes in both therapeutic eras (Table S2, Figures S1A, S2A-C).

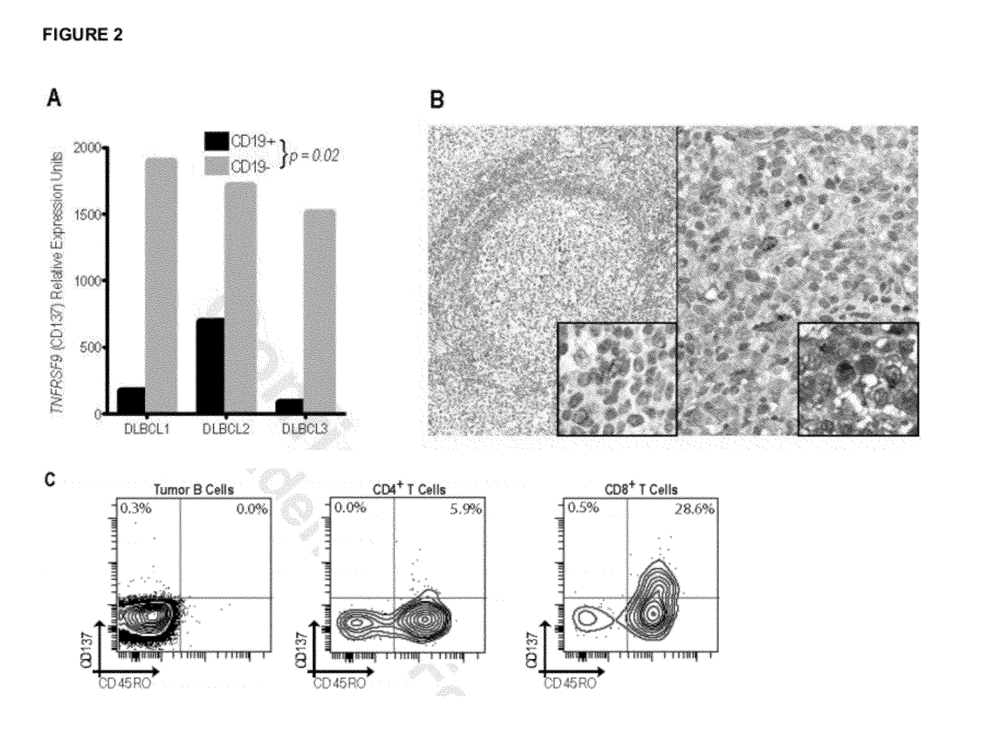

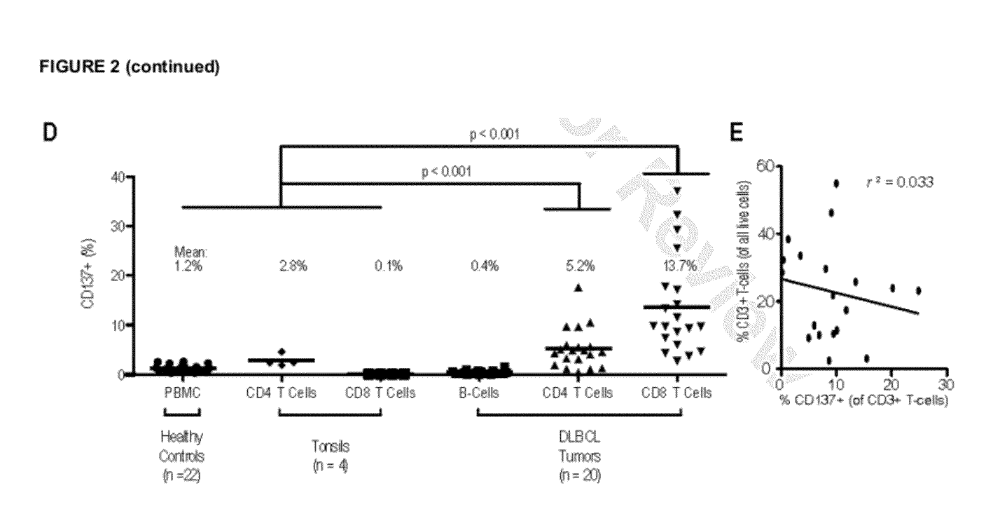

TNFRSF9 EXPRESSION IS LIMITED TO A TUMOR INFILTRATING CELL SUBSET. We observed higher expression of TNFRSF9 mRNA on non-tumor cells than paired lymphoma B cells (p<0.05, FIG. 2A). Given its expression as a marker of activated T and NK cells that commonly infiltrate many tumors, we examined TNFRSF9 expression in publicly available gene expression data across tumors from diverse histologies (n=1822), and confirmed significantly higher expression among lymphomas than other tumor types (p<0.0001, Figure S3). Within DLBCL, we verified limited distribution of expression of the encoded protein using immunohistochemistry, finding CD137 exclusively within rare tumor infiltrating cells (FIG. 2B). Cell surface immunophenotyping also demonstrated no significant CD137 expression on tumor cells (mean: 0.4%, range: 0-1.6%, FIG. 2C-D).

Among tumor infiltrating T-cells, significant but varying frequencies of CD8 and CD4 cells expressed CD137, mainly within a minor subset with a memory (CD45RO+) phenotype (FIG. 2C). Indeed, this activated memory phenotype distinguished infiltrating T-cell subsets of DLBCL tumors from healthy lymphoid tissues (FIG. 2D), suggesting unique interactions within the DLBCL microenvironment. Notably, TNFRSF9 expression was not simply a proxy for infiltrating T-cell frequency, as no significant correlation was observed between them (FIG. 2E). Rather, variation in TNFRSF9 mRNA in DLBCL likely reflects frequency of an activated T-cell subset (Figure S4).

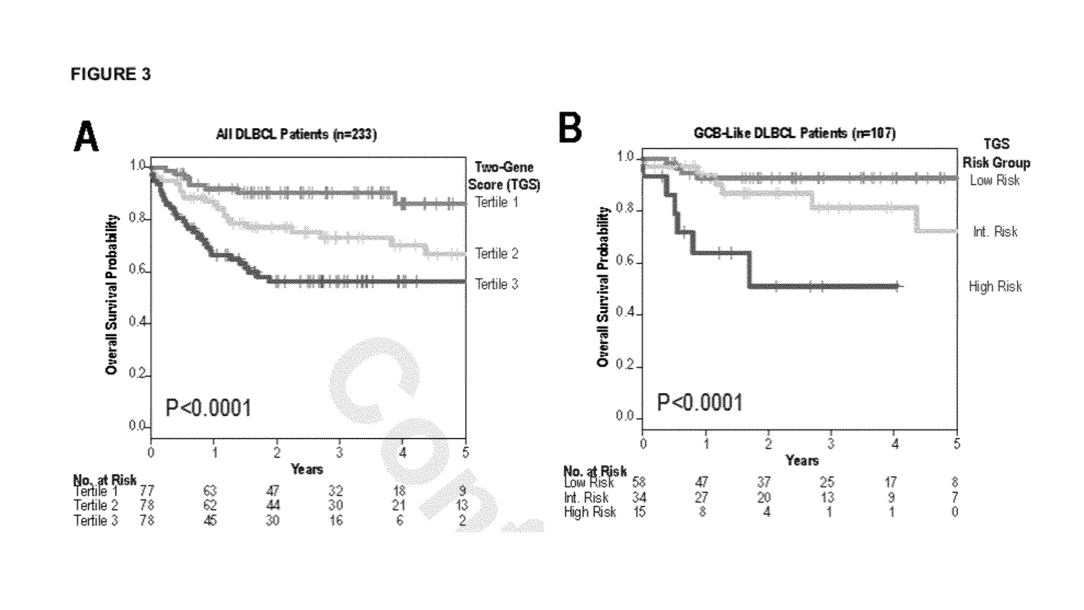

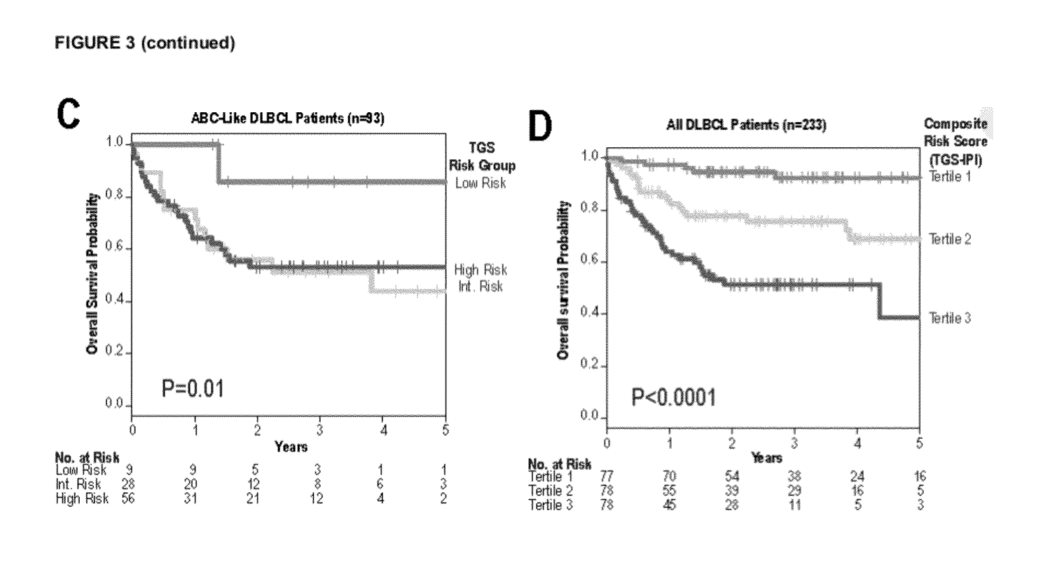

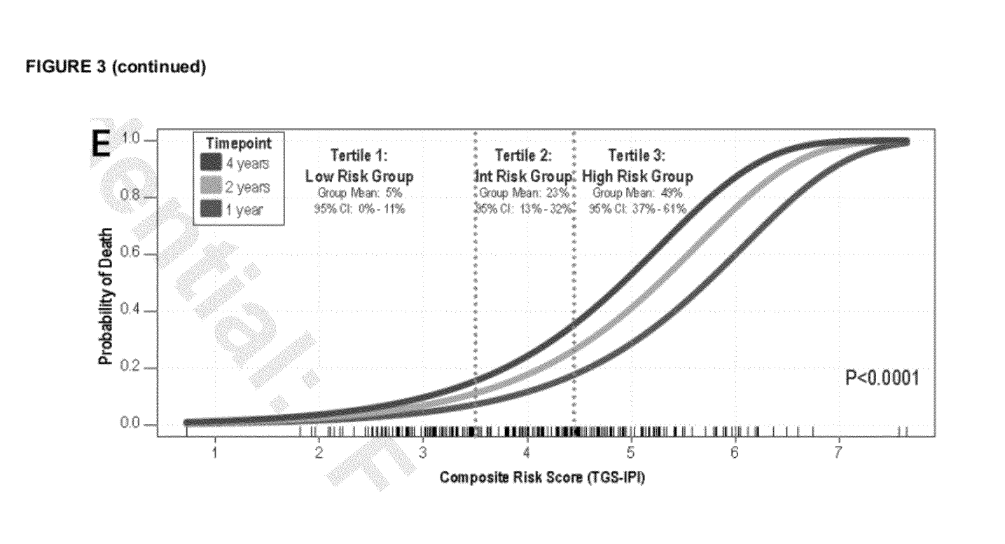

A TWO GENE MODEL IS A SIGNIFICANT DETERMINANT OF SURVIVAL. We next examined the prognostic strength of the bivariate model combining expression of the tumor biomarker LMO2, with the microenvironment marker TNFRSF9, weighting the two genes based on their independent contributions from multivariate Cox regression in the training cohort (DLBCL1). We thus defined a two gene score [TGS=(−0.32×LMO2)+(−0.29×TNFRSF9)] that effectively predicted overall survival in patients treated with R-CHOP (p<0.0001; FIG. 3A-C). For each incremental unit rise of the TGS, there was a 2.7-fold (95% Cl, 2.0 to 3.8) increase in relative risk of death. Tertiles of the TGS stratified patients with distinct outcomes (Figure S5), with corresponding 2-year overall survival rates of 56%, 77%, and 91% (FIG. 3A). Importantly, in multiple independent cohorts (DLBCL2-4) we validated the model and previously defined thresholds from the training set (Figure S6A-B).

TGS CARRIES INDEPENDENT PROGNOSTIC VALUE. When assessed within DLBCL subtypes, high TGS scores identified patients with relatively adverse outcomes among the more favorable GCB-like variant (FIG. 3B). Conversely, within the less favorable ABC-like tumors, low scores identified patients with superior outcomes (FIG. 3C). Within specific IPI risk groups, the TGS similarly identified patients with discordant outcomes in both therapeutic eras (Figure S7). In multivariate analyses, the TGS was also independent of the International Prognostic Index (IPI), and a ‘stromal’ model comprising 381 genes that integrates cell-of-origin (Table S3). Furthermore, the combination of the two genes compared favorably to other previously described models comprising 6-genes or 381-genes, both of which also included LMO2 but not TNFRSF9 (Figure S8).

A COMPOSITE MODEL INTEGRATING TGS WITH IPI. Given that the TGS added significantly to IPI, we constructed a combined model integrating both indices. We derived a composite score (TGS-IPI) for patients treated with R-CHOP in the training cohort, weighting the IPI score (on a 0-5 scale) and TGS based on their independent contributions from multivariate Cox regression [TGS-IPI=(0.93×TGS)+(0.6×IPI)+4]. This composite score significantly outperformed the TGS or IPI alone in predicting survival in independent cohorts. Tertiles of the TGS-IPI also separated patients into three significantly different strata (FIG. 3D-E). Patients in corresponding risk groups [i.e., High Risk, TGS-IPI>4.51; Intermediate Risk, 4.51εTGS>3.47; Low Risk, TGS≦3.47] had estimated 2-year overall survival rates of 51%, 78%, and 95%, respectively. Importantly, by applying the previously defined model and thresholds, these findings were validated in independent cohorts (Figure S6C). To visualize how these groups relate to the distribution of continuous TGS-IPI, we modeled risk of death as a function of the TGS-IPI (FIG. 3E).

EXTERNAL CLINICAL VALIDATION OF SURVIVAL MODEL. While the TGS and TGS-IPI were validated in multiple cohorts when LMO2 and TNFRSF9 were measured using microarrays, this technique is not yet widely available in clinical laboratories. Therefore, we used quantitative real time polymerase chain reaction to measure the expression of LMO2 and TNFRSF9 in diagnostic formalin fixed, paraffin embedded samples from an independent set of 147 patients with DLBCL treated with R-CHOP (DLBCL4). We also created a publicly available calculator to simplify estimation of risk using the TGS and TGS-IPI on fixed specimens.

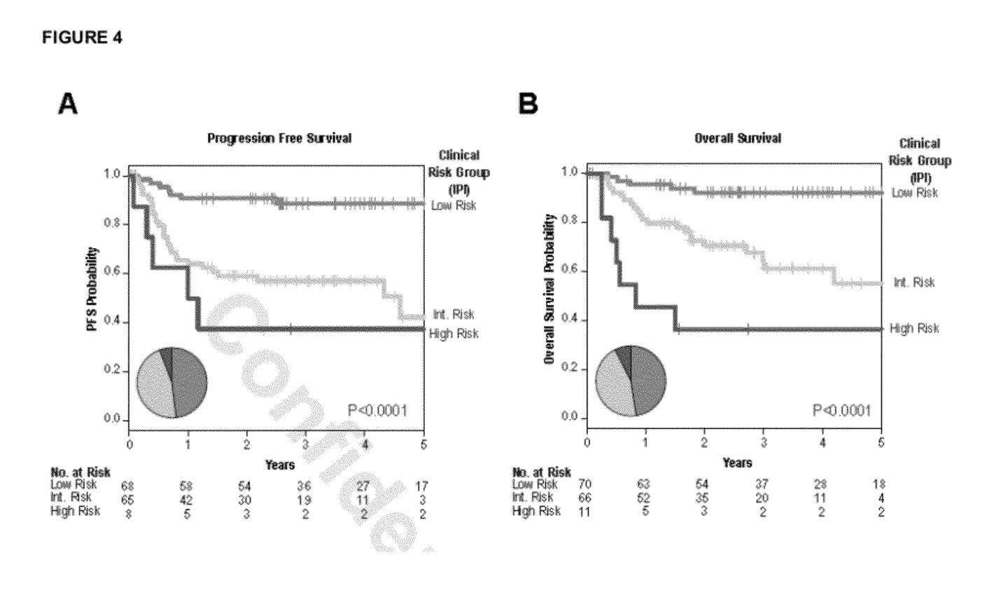

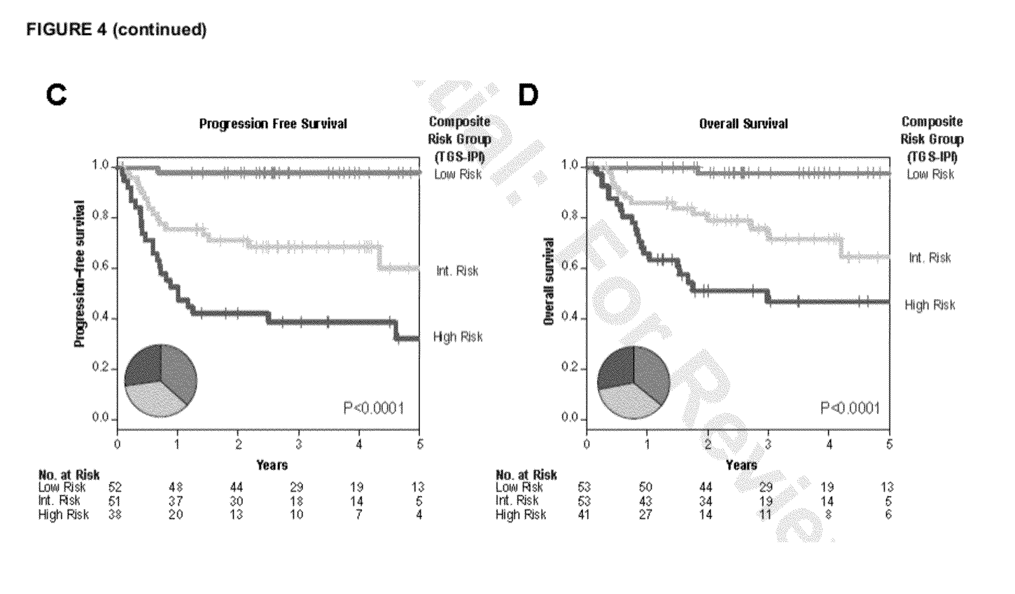

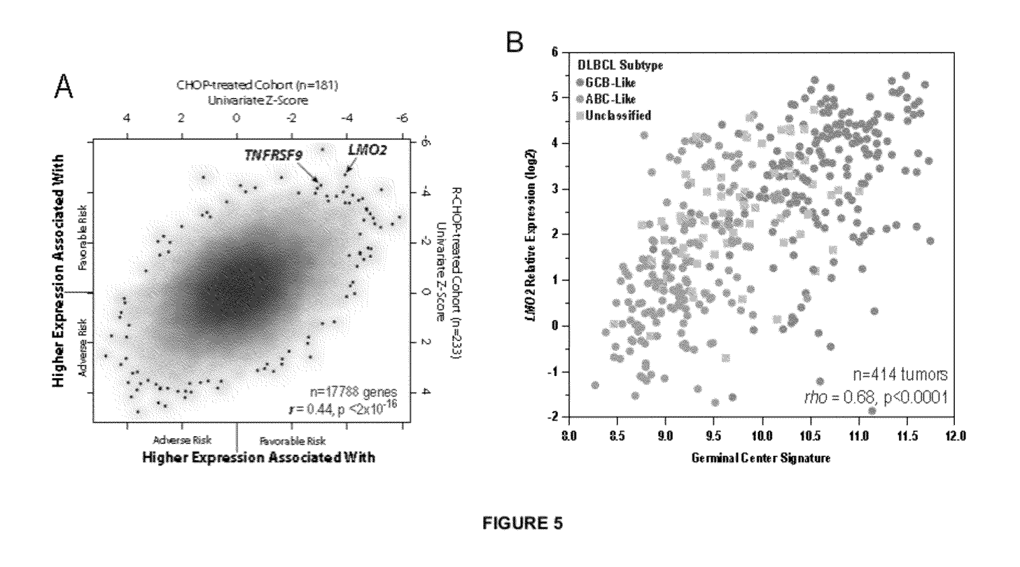

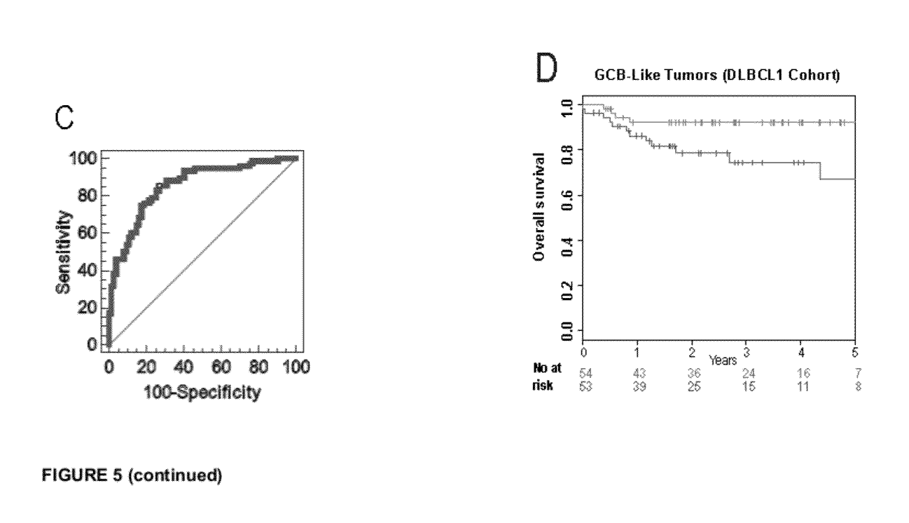

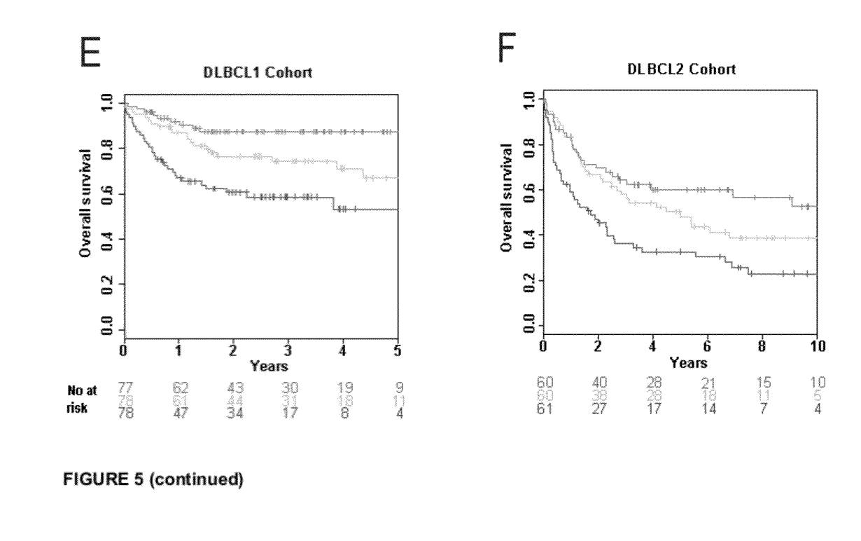

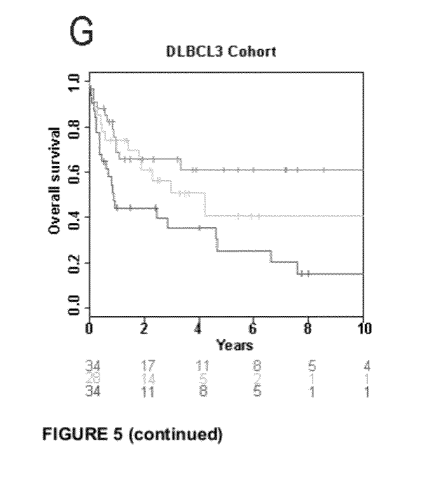

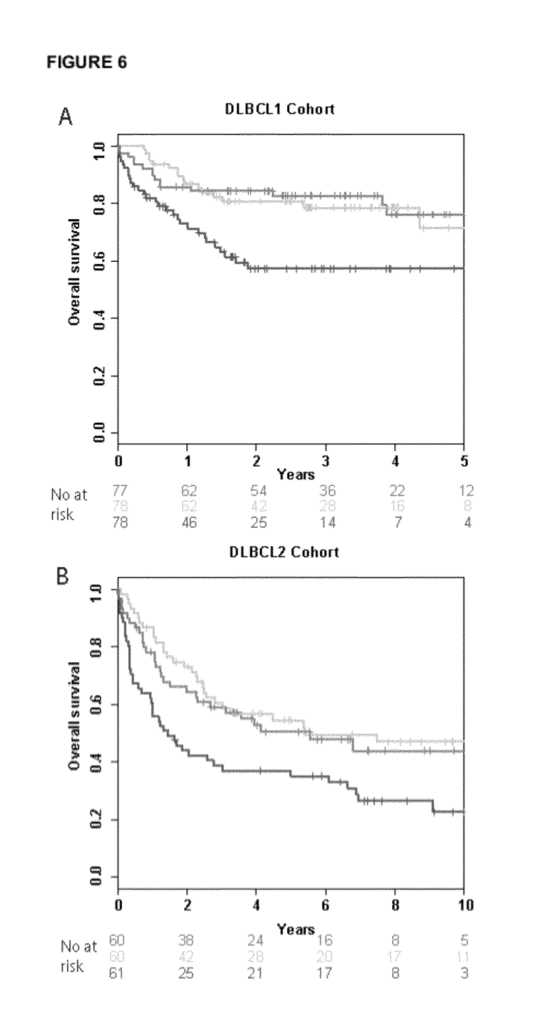

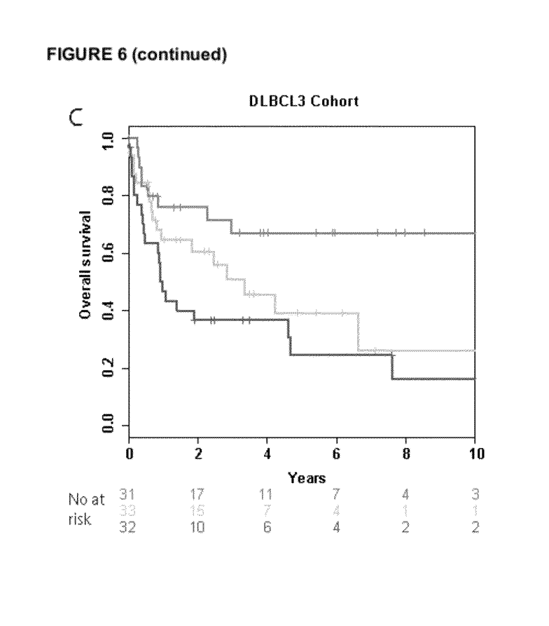

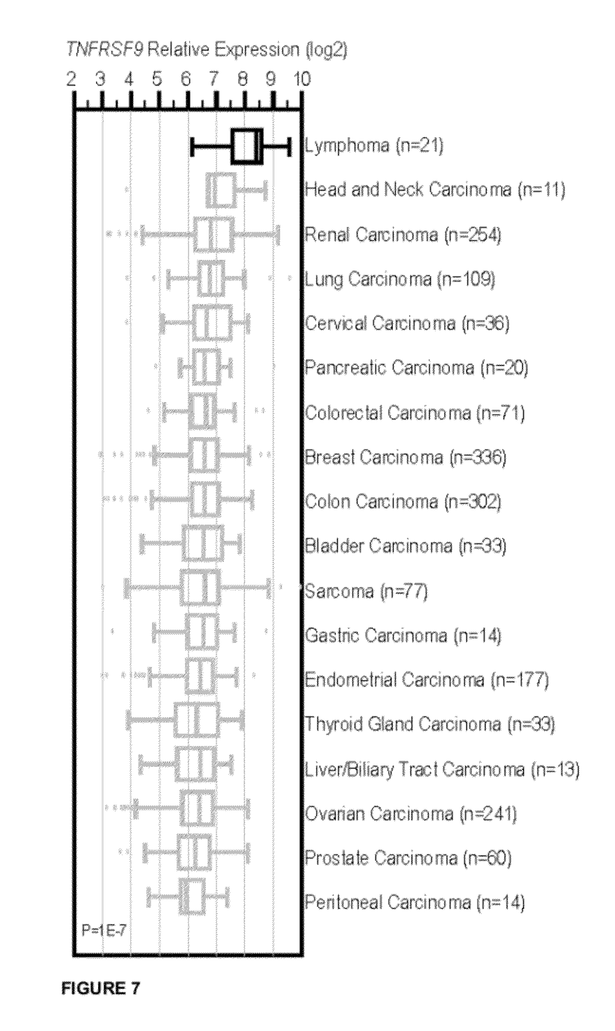

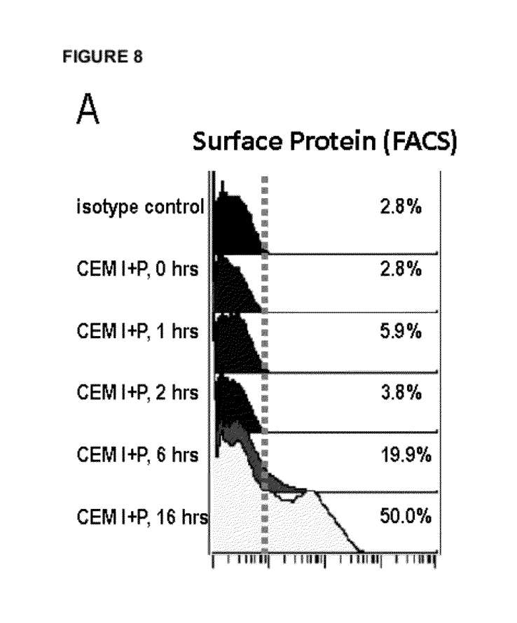

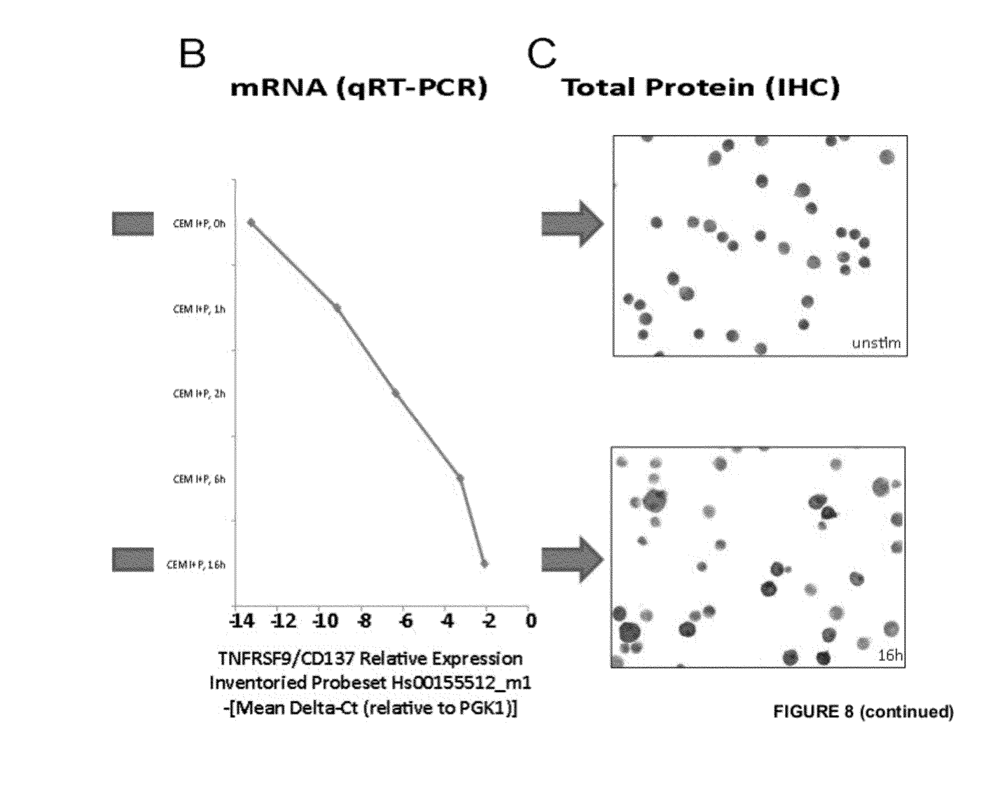

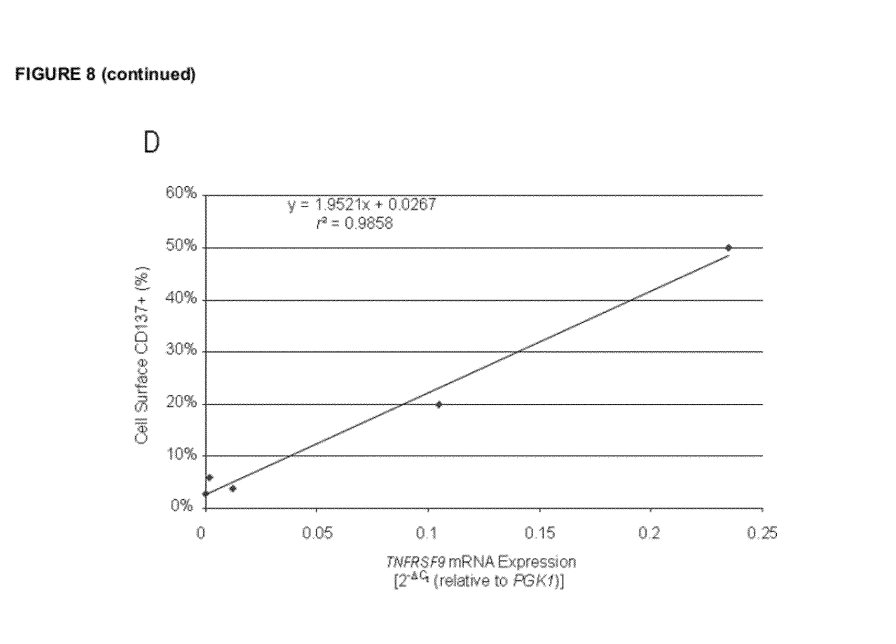

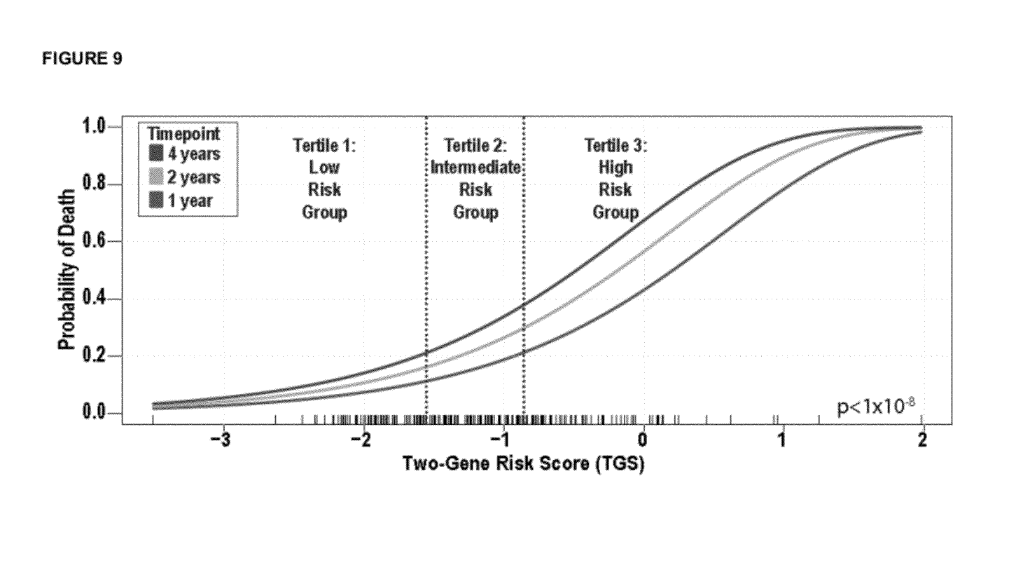

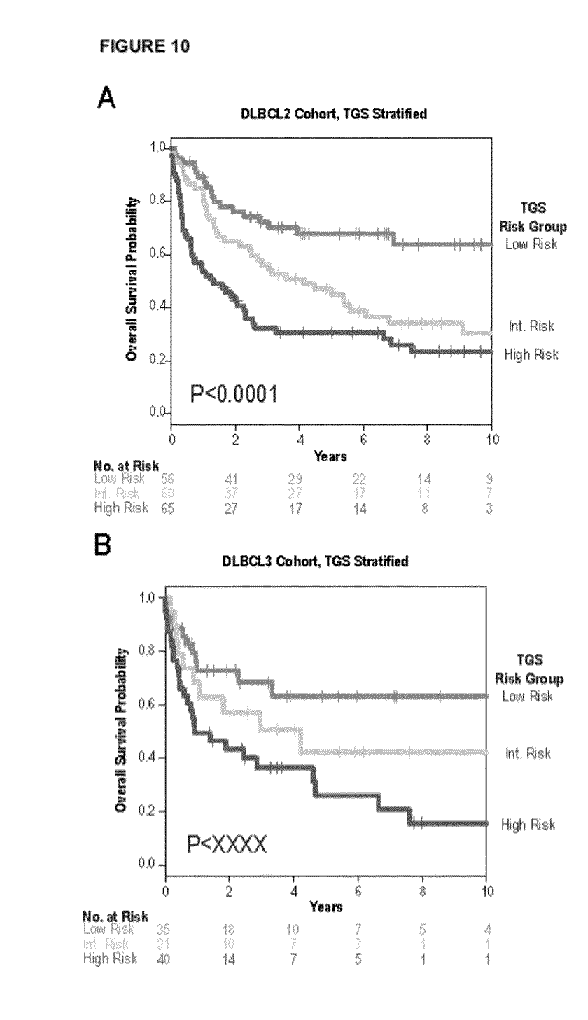

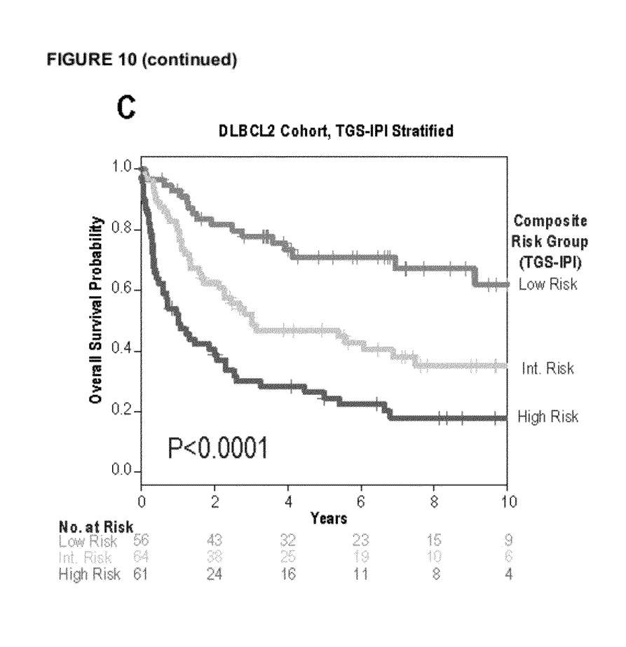

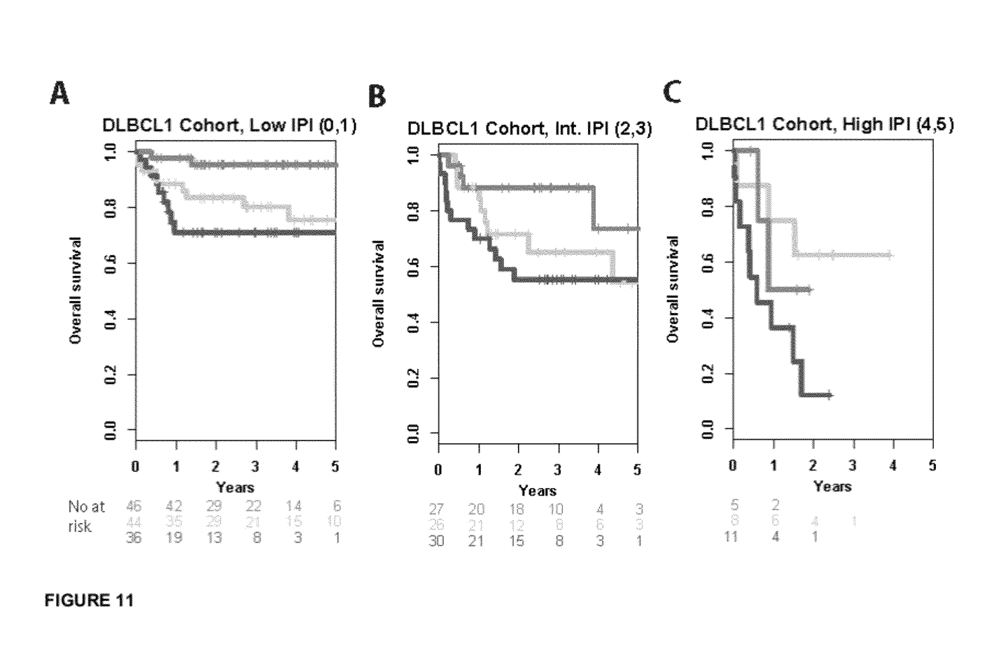

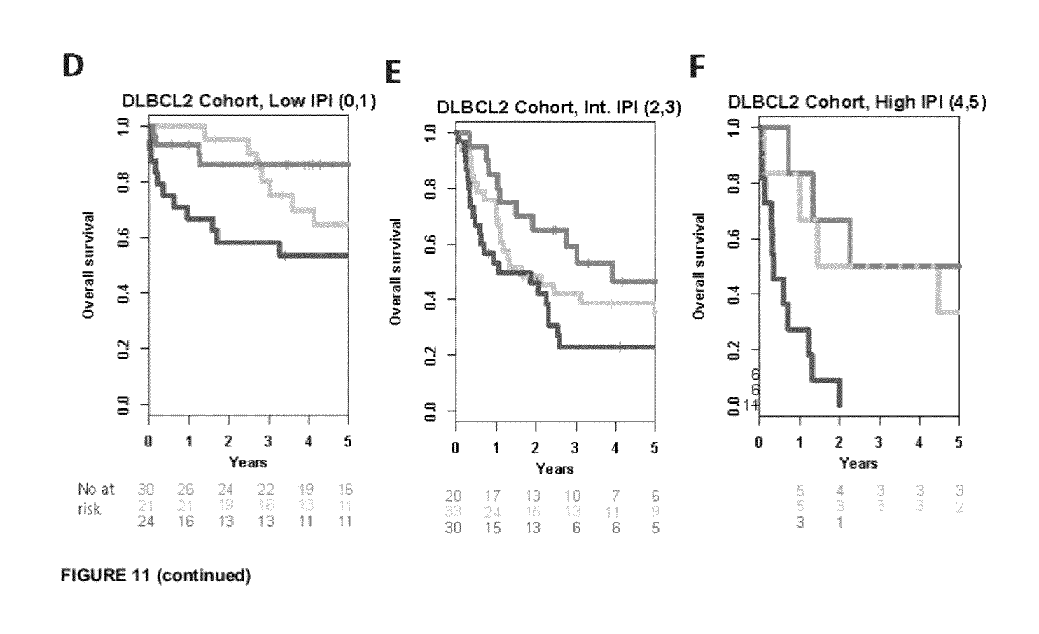

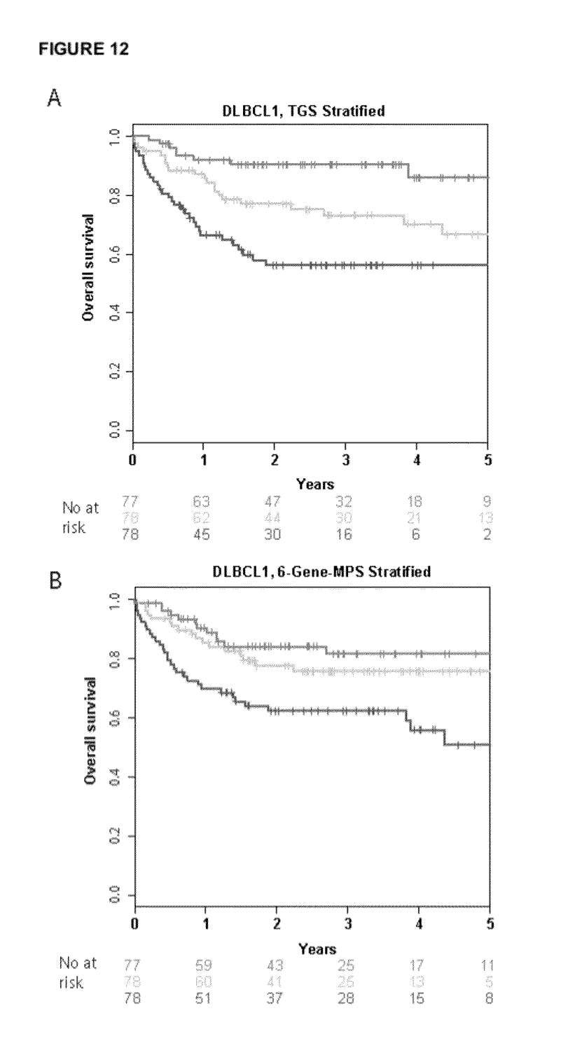

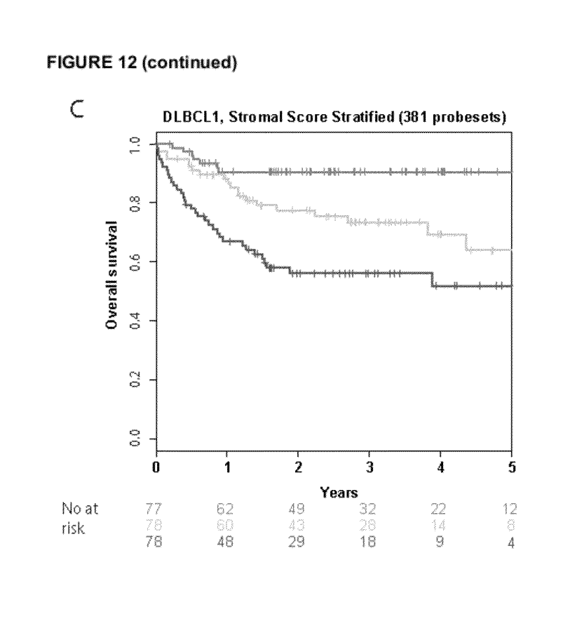

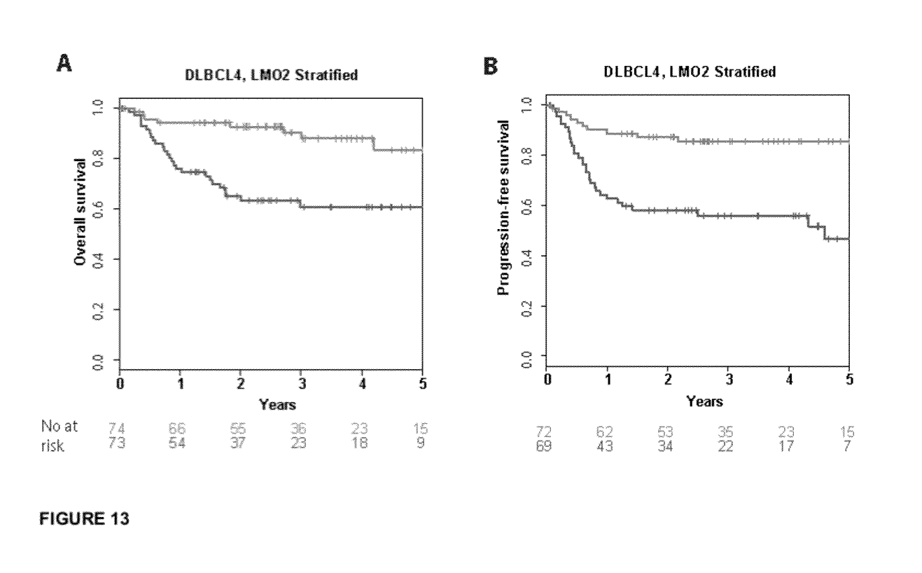

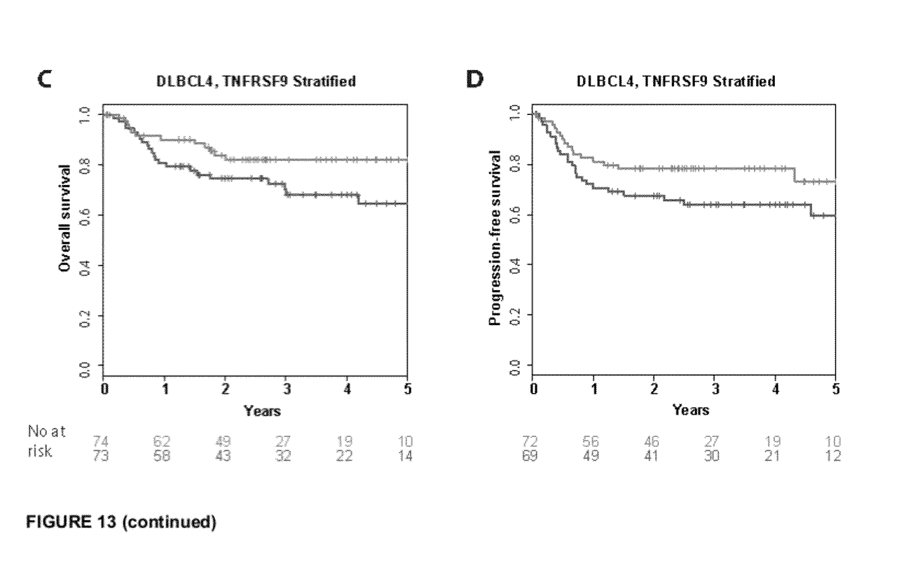

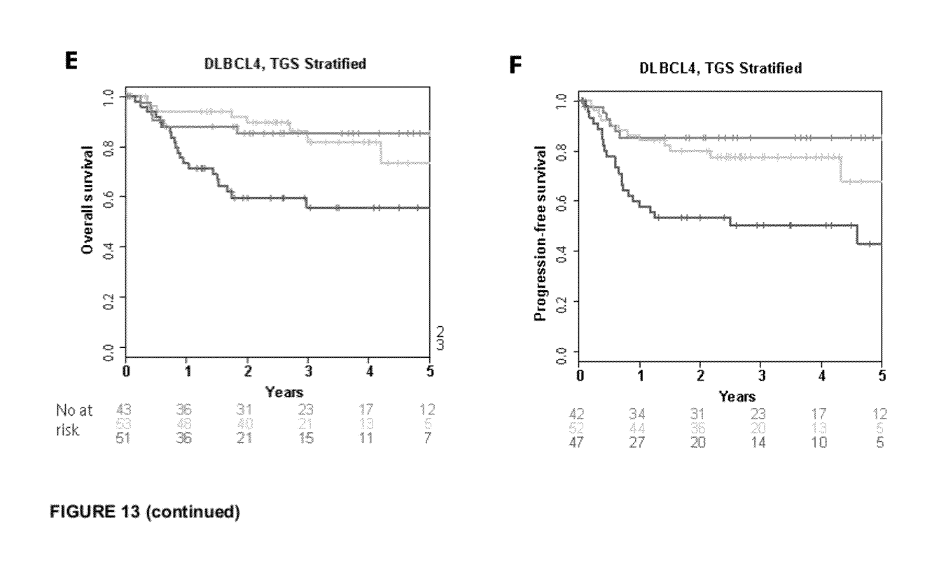

In univariate and multivariate analyses of this cohort, LMO2 and TNFRSF9 expression remained prognostic of both overall (OS) and progression free survival (PFS) (Figure S9A-D), and when combined, the previously defined thresholds of the TGS and TGS-IPI stratified groups with distinct OS and PFS (FIGS. 4C-D and S9E-F). Within this validation cohort, the TGS and IPI remained independent predictors of OS and PFS (Table S4). When combined as a continuous score, the TGS-IPI was predictive of both OS and PFS (p<0.0001, HR 2.8, 95% Cl 2.0-3.8, and p<0.0001, HR 2.6, 95% Cl 1.9-3.4, respectively).

Stratification into low-, intermediate- and high-risk groups using predefined TGS-IPI thresholds separated groups with distinct PFS and OS (FIG. 4C-D). Patients in corresponding risk groups had estimated 2-year progression free survival rates of 98%, 71%, and 42%; the 2-year overall survival rates for these groups were 98%, 79%, and 51%, respectively (P<0.001). Importantly, for both PFS and OS, TGS-IPI captured a larger subset of patients at extremes of risk, particularly for those at high risk of death following R-CHOP chemotherapy (FIG. 4).

Despite significant scientific progress made possible by the human genome project, corresponding advances in clinical medicine have been relatively modest. Current genome-scale studies have provided a rich source of molecular data that can be correlated with outcomes. Prior efforts to leverage transcriptome profiles have often employed average representations of co-regulated genes as composite signatures or ‘metagenes’. However, the need to measure many genes poses practical barriers to external validation and clinical application. Further, the requirement for unfixed diagnostic tissues has limited the clinical utility of such methods. In the case of DLBCL, variation in outcomes suggests that clinical features cannot fully account for underlying biological heterogeneity. Molecular profiling studies have attempted to capture this diversity, for instance by defining subtypes relating to cell of origin, such as Germinal Center B-Cell Like and Activated B-Cell Like types. Multivariate predictive models integrating these subtypes have been proposed, capturing additional features from the tumor microenvironment. Unfortunately, surrogate methods employing fewer genes have had conflicting performance in their prognostic influence, and such methods are unable to classify 15-50% of patients with DLBCL, limiting clinical utility. We employed an alternative strategy, by leveraging existing knowledge and available data, identifying LMO2 and TNFRSF9 as two key genes whose expression each provides independent prognostic value.

Expression of the LMO2 transcription factor is a marker for germinal center B-cell differentiation stage. Over expression of LMO2 among nearly 10% of T-cell acute lymphoblastic leukemias can be ascribed to several recurrent genetic alterations, though no significant corresponding prognostic value has been observed in T-ALL. Nevertheless, over-expression of LMO2 is associated with induction and promotion of self-renewal within committed lymphocytes en route to leukemia in mice and also in humans, suggesting a similar role in lymphomagenesis.

TNFRSF9 encodes the co-stimulatory receptor CD137, with expression largely limited to activated T and NK cells wherein it plays important roles in immunological memory formation and effector functions. Our observation of higher CD137 expression on a minor subset of infiltrating non-neoplastic cells of DLBCL tumors likely reflects unique interactions within the local microenvironment. Indeed, resting immune effector cells from peripheral blood could be induced to express high levels of CD137 after contact with tumor cells, an effect that could be augmented by rituximab.

Notably, we found no significant relationship between expression of TNFRSF9 on infiltrating T cells and reciprocal regulatory molecules on tumor cells including its ligand TNFSF9, or class II genes from the major histocompatibility loci, both of which have been implicated previously in lymphomas. Thus, the basis for observed variation in frequency of CD137 expressing infiltrating T-cells in DLBCL is unclear. Agonistic monoclonal antibodies against CD137 have potent immunoregulatory properties and can eradicate tumors in multiple pre-clinical models including lymphomas. Therapeutic targeting of this molecule is the subject of ongoing clinical trials. The higher expression of CD137 observed among lymphomas, as compared to solid tumors, may represent a unique therapeutic opportunity in this disease. Also, CD137 expression on infiltrating immune cells in DLBCL might serve as a predictive biomarker for response to a monoclonal antibody, such as anti-CD137, that triggers effector anti-tumor functions. Since higher expression of CD137 confers a good prognosis for DLBCL patients, such therapeutic targeting with monoclonal antibodies could be a means of limiting chemotherapy with its attendant toxicities for this subgroup of patients.

While the contribution of gene expression signatures from the tumor microenvironment has previously been recognized as an important prognostic factor among diverse lymphomas, to our knowledge this is the first such report for TNFRSF9. Notably, TNFRSF9 was not included on Lymphochip DNA microarrays employed in earlier DLBCL profiling studies. A more recent study from the Leukemia and Lymphoma Molecular Profiling Project (LLMPP) also did not identify TNFRSF9, since that study focused on signatures where higher expression was associated with adverse outcomes. Finally, the TGS and TGS-IPI are distinguished from prior prognostic models, in that their weighting of component risk factors were both derived and validated in the current therapeutic era that includes rituximab.

The newly described bivariate model and a composite index integrating the IPI were reproducible within validation studies, including in a simple assay employing routinely obtained diagnostic pathological specimens that were formalin fixed and paraffin embedded. While IPI captures a significant portion of attributable risk for adverse outcomes even in the current era,₅ the TGS carried independent value and obviated the need for more complex multi-gene indices. Both the TGS and the IPI added to each other within the TGS-IPI, capturing a larger fraction of patients with adverse outcomes.

Though originally devised prior to introduction of rituximab, the components of the IPI can be used to predict extremes of outcomes in the current therapeutic era. For instance, patients with zero IPI risk factors have 4-year survivals estimated at 95% in the current era but comprise only 10-15% of unselected cohorts. In comparison, the TGS-IPI identified over 30% of patients exhibiting similar outcomes after therapy with R-CHOP. Conversely, whereas patients with adverse risk as defined by TGSIPI comprise at least 25% of the R-CHOP treated patients, the corresponding IPI High Risk group captures only ˜10% of patients. Therefore, by capturing more patients at low and high risk for progression and death, this composite index allows better risk assignment than either the IPI or TGS alone. Since less than half of patients within the high TGS-IPI risk group are cured, novel strategies to improve their outcomes are urgently needed, incorporating additional therapies or alternative regimens to RCHOP. Conversely, given their highly favorable risk profile, patients with good risk features are unlikely to benefit from therapies adding to R-CHOP; similar strategies could be used to guide trials aiming to minimize toxicities from chemotherapy in this group.

TABLE 2

| Overall Survival, Covariate | HR (95% CI) | P |
|---|---|---|
| LMO2 expression | 0.8 (0.6-0.9) | 0.009 |
| Germinal Center Signature | 0.7 (0.5-1.1) | 0.15 |
| Overall | | <0.0001 |

LMO2 expression retains prognostic value in DLBCL when considering it alongside the germinal center signature, as covariates in a multivariate model for overall survival after R-CHOP chemotherapy (DLBCL1 dataset, as specified within Table 1).

TABLE 3

| | | Association with overall | | | Tumor vs. |
|---|---|---|---|---|---|
| | | survival in RCHOP | | | Micro- |
| | | training cohort (DLBCL1) | | | Environment |
| | | | | Bi- | (CD19+ vs |
| | | | | variate | CD19−) |
| Entrez | | Uni- | | (with | p-value |
| Gene | Gene | variate | | LMO2) | (paired |
| Identifier | Symbol | z-score | p-value | p-value | T-test) |
| 3604 | TNFRSF9 | −4.30 | 1.72E−05 | 2.33E−08 | 0.04 |
| | (CD137) | | | | |
| 5578 | PRKCA | −3.21 | 1.32E−03 | 1.04E−07 | 0.01 |
| 5334 | PLCL1 | −3.22 | 1.29E−03 | 1.67E−07 | 0.01 |
| 64375 | IKZF4 | −3.71 | 2.09E−04 | 1.93E−07 | 0.04 |
| 80273 | GRPEL1 | 3.23 | 1.24E−03 | 1.06E−06 | 0.00 |
| 81615 | TMEM163 | −3.09 | 2.03E−03 | 1.17E−06 | 0.02 |
| 56548 | CHST7 | 3.80 | 1.44E−04 | 1.46E−06 | 0.04 |
| 113763 | C7orf29 | −3.15 | 1.61E−03 | 1.65E−06 | 0.00 |
| 321 | APBA2 | −3.05 | 2.26E−03 | 1.74E−06 | 0.05 |
| 3687 | ITGAX | −3.70 | 2.20E−04 | 2.33E−06 | 0.01 |
| 286133 | SCARA5 | −2.60 | 9.36E−03 | 3.65E−06 | 0.05 |
| 5919 | RARRES2 | −2.74 | 6.07E−03 | 4.58E−06 | 0.01 |
| 2060 | EPS15 | −3.22 | 1.29E−03 | 5.04E−06 | 0.03 |
| 6355 | CCL8 | 2.76 | 5.74E−03 | 6.25E−06 | 0.01 |
| 4208 | MEF2C | −2.75 | 5.92E−03 | 1.45E−05 | 0.01 |

TNFRSF9 as best univariate and bivariate predictor among genes more highly expressed in non-tumor cells. Bivariate combinations of genes with LMO2 were tested for their ability to predict survival using multivariate Cox regression, limited to genes with higher expression among non-tumor cells (CD19−) as compared with paired t-test (p < 0.05) to matched tumors cells
(CD19+), and exhibiting significant (p < 0.01) univariate predictive value in R-CHOP treated patients (DLBCL1). Within this group of genes, TNFRSF9 was best as a univariate predictor and also as a bivariate partner for LMO2. The top 15 genes are shown, ranked by p-value of the bivariate model.

TABLE 4

| Overall Survival, Covariates | H.R. (95% CI) | P |
|---|---|---|
| IPI Score (0-5) | 1.8 (1.4-2.2) | <0.0001 |
| Two Gene Risk Score (TGS) | 2.3 (1.4-3.8) | 0.001 |
| Stromal Score (381 genes) | 1.1 (0.7-2.0) | 0.59 |
| Overall | | <0.0001 |

Multivariate analysis of overall survival after R-CHOP therapy (DLBCL1 microarray dataset). In a multivariate model considering IPI, TGS, and Stromal score (comprising the Germinal center signature, Stromal1 and Stromal2 signatures), only the IPI and TGS remain significant predictors of OS.

TABLE 5

| | Overall Survival | | Progression Free Survival | |
|---|---|---|---|---|
| Covariate | HR (95% CI) | P | HR (95% CI) | P |
| Two Gene Risk | 1.9 (1.2-3.1) | 0.006 | 2.2 (1.4-3.4) | 0.0003 |
| Score (TGS) | | | | |
| IPI Score (0-5) | 2.2 (1.7-3.0) | <0.0001 | 1.8 (1.4-2.4) | <0.0001 |
| Overall | | <0.0001 | | <0.0001 |

In a multivariate analysis for outcomes after R-CHOP therapy within the external validation cohort (DLBCL4 dataset), both TGS and IPI remain as independent predictors of OS and PFS.

What is claimed is:

1. A method of providing a prognosis, or a prediction of responsiveness to therapy for a patient with a Non-Hodgkin lymphoma (NHL), the method comprising:
obtaining an expression representation for a biopsy sample from said lymphoma, representing the expression level of LMO2 and TNFRSF9; and
employing the expression representation to provide a prognosis or determination of a therapeutic treatment for said patient.

2. The method of claim 1 wherein the expression representation represents the expression level of LMO2 by tumor cells and TNFRSF9 by the immune microenvironment of the tumor.

3. The method according to claim 1, wherein the expression representation is two gene score (TGS), wherein the TGS is calculated from the normalized expression level of each of said one or more genes in a reference dataset.

4. The method according to claim 1, wherein the expression representation is two gene score (TGS), wherein the TGS is calculated from the weighted normalized expression level of each of said one or more genes in a reference dataset.

5. The method of claim 1, further comprising the step of integrating the TGS with an international prognosis index (IPI) for said patient.

6. The method according to claim 3, wherein the NHL is a diffuse large B cell lymphoma (DLBCL).

7. The method according to claim 1, wherein said employing step comprises comparing said TGS to the TGS of one or more reference samples.

8. The method according to claim 1, wherein said disease prognosis is a prognosis of overall survival (OS), progression-free survival (PFS); relapse-free survival (RFS) and/or event-free survival (EFS).

9. A kit for use in providing a prognosis, or a determination of a therapeutic treatment for a patient with NHL, the kit comprising:
reagents to obtain an expression representation as set forth in claim 1 from a tumor biopsy sample from a patient; and
an expression representation reference.

10. A method of treating a person with NHL, comprising:
obtaining an expression representation for a biopsy sample from said lymphoma, representing the expression level of LMO2 and TNFRSF9; and
employing the expression representation to provide a prognosis or determination of a therapeutic treatment for said patient; and
treating said person with appropriate chemotherapy and/or immunotherapy.

11. The method of claim 10 wherein the expression representation represents the expression level of LMO2 by tumor cells and TNFRSF9 by the immune microenvironment of the tumor.

12. The method according to claim 10, wherein the expression representation is two gene score (TGS), wherein the TGS is calculated from the normalized expression level of each of said one or more genes in a reference dataset.

13. The method according to claim 10, wherein the expression representation is two gene score (TGS), wherein the TGS is calculated from the weighted normalized expression level of each of said one or more genes in a reference dataset.

14. The method of claim 10, further comprising the step of integrating the TGS with an international prognosis index (IPI) for said patient.

15. The method according to claim 10, wherein the NHL is a diffuse large B cell lymphoma (DLBCL).

16. The method according to claim 10, wherein said employing step comprises comparing said TGS to the TGS of one or more reference samples.

17. The method according to claim 10, wherein said chemotherapy and/or immunotherapy is provided in conjunction with an inducible costimulatory molecule agonist.

18. The method according to claim 10, wherein said inducible costimulatory molecule agonist is anti-CD137.
